(19) United States
(12) Patent Application Publication
(10) Pub. No.: US 2024/0279702 A1
(43) Pub. Date: Aug. 22, 2024

(54) SOYBEAN AND MAIZE CELL-FREE EXPRESSION SYSTEMS

(71) Applicants: CORTEVA AGRISCIENCE LLC, INDIANAPOLIS, IN (US); FRAUNHOFER-GESELLSCHAFT ZUR FÖRDERUNG DER ANGEWANDTEN FORSCHUNG EV, MÜNCHEN (DE)

(72) Inventors: MATTHIAS BUNTRU, AACHEN (DE); KRISHNA MADDURI, CARMEL, IN (US); STEFAN SCHILLBERG, AACHEN (DE); SIMON VOGEL, AACHEN (DE)

(73) Assignees: CORTEVA AGRISCIENCE LLC, INDIANAPOLIS, IN (US); FRAUNHOFER-GESELLSCHAFT ZUR FÖRDERUNG DER ANGEWANDTEN FORSCHUNG EV, MÜNCHEN (DE)

(21) Appl. No.: 18/569,001

(22) PCT Filed: Jun. 29, 2022
(86) PCT No.: PCT/US2022/035544

§ 371 Date: Dec. 11, 2023

Related U.S. Application Data
(60) Provisional application No. 63/216,854, filed on Jun. 30, 2021.

(51) Int. Cl. C12P 21/00; C07K 1/14; C12N 5/04; C12Q 1/6895

(52) U.S. Cl. CPC C12P 21/00; C07K 1/14; C12N 5/04; C12Q 1/6895; C12Q 2600/13

(57) ABSTRACT

This disclosure concerns the systems, methods, and kits for the in vitro synthesis of biological macromolecules in a reaction utilizing maize or soybean cell lysates.

### CROSS-REFERENCE TO RELATED APPLICATIONS

This application claims the right of priority and benefit of U.S. Provisional Application 63/216,854 filed on Jun. 30, 2021, the contents of which are incorporated by reference herein in their entirety.

### FIELD OF THE DISCLOSURE

The present disclosure relates to the in vitro production of biopolymers. Some embodiments relate to the production of, for example, polypeptides, polynucleotides, and/or polysaccharides in soybean and maize cell-free systems.

### BACKGROUND

Cell-free expression systems are preferred over conventional in vivo protein expression systems for use in a variety of applications. For example, many biopolymers are difficult to produce, unstable, toxic, or susceptible to lytic degradation when expressed in living cells. Furthermore, the purification of biopolymers from prokaryotic or eukaryotic host cells can present safety concerns, e.g., when host cells are not generally recognized as safe and/or when it is difficult to isolate the biopolymer from potential toxins, allergens, or other impurities present in host cells. Thus, in some cases cell-free expression systems are desirable for use in the production of pharmaceutical or food products.

In the agricultural field, expression of transgene products (e.g., RNA, siRNA, gene editing machinery, and proteins) in crop plants can provide critical benefits to farmers. For example, transgenic proteins provide for increased crop yields, use of safer and/or more effective herbicides, reduced need for insecticide use, and soil conservation by reducing or eliminating the need for tilling. However, transgene expression in plants is a complex process. Proper expression of transgenic products, including at desirable levels, is influenced by various complex processes that include gene transcription, translation, protein folding, glycosylation, and phosphorylation. Each of these processes depends on different enzymes, functional group donors, and cofactors in a plant cell. The combined effect of these can be unpredictable and variable in different plant types. Thus, to bring about the optimal expression of a functional transgenic gene product appropriate transcriptional elements such as enhancers and promoters must be selected, transgene coding sequences are frequently be altered (e.g., by codon-optimization), non-coding intron sequences can be added, subtracted, or modified, and the ability of a plant cell to properly fold and modify newly translated proteins, e.g., by attaching glycans or phosphates, must be considered and evaluated to determine.

Compared to transformed plant or tissue-based expression, cell-free protein expression plant systems (“CFPS”) offers advantages such as shorter process times and the direct control and monitoring of reaction conditions. Swartz (2012), supra. Thus, CFPS make it simpler and faster to screen large number of variables to determine which factors enable or improve transgenic gene expression, without (or prior to) creating transgenic tissues/plants, which can be a labor-intensive, time-consuming, and space-demanding process. For example, PCR products can be used directly for the simultaneous expression of multiple proteins without laborious cloning and transformation steps. Wu et al. (2007) Angew. Chem. Int. Ed. Engl. 46(18):3356-8; Yabuki et al. (2007) J. Struct. Funct. Genomics 8(4):173-91; Gan & Jewett (2014) Biotechnol. J. 9(5):641-51. CFPS platforms allow the addition of accessory factors that promote protein folding (Ozawa et al. (2005) J. Biomol. NMR 32(3):235-41; Endo et al. (2006) Mol. Biotechnol. 33(3):199-209; Matsuda et al. (2006) J. Struct. Funct. Genomics 7(2):93-100). They also facilitate the expression of cytotoxic proteins that cannot be produced in living cells. Xu et al. (2005) Appl. Biochem. Biotechnol. 127(1):53-62; Schwarz et al. (2008) Proteomics 8(19):3933-46; Xun et al. (2009) Protein Expr. Purif 68(1):22-7.

Escherichia coli cell-free lysates are widely used and are advantageous because of their low cost, scalability, and high productivity. Zawada et al. (2011) Biotechnol. Bioeng. 108(7):1570-8; Caschera & Noireaux (2014) Biochimie 99:162-8. However, because the lysates originate from bacteria, they are unsuitable for the production of complex proteins with multiple subdomains due to inefficient oxidative folding, and the absence of chaperones and glycosylation machinery. Eukaryotic cell-free systems are better suited for the expression of such proteins, and support most forms of post-translational modification. Chang et al. (2005) J. Mol. Biol. 353(2):397-409; Zhang & Kaufman (2006) Handb. Exp. Pharmacol. (172):69-91. The most frequently used systems are based on wheat germ extract (WGE), insect cell extract (ICE), and rabbit reticulocyte lysate (RLL). However, these systems are expensive, and extract preparation is complex. Carlson et al. (2012), supra. This has created a demand for additional eukaryotic CFPS, such as those based on Leishmania tarentolae (Mureev et al. (2009) Nat. Biotechnol. 27(8):747-52), Chinese hamster ovary (CHO) cells (Brodel et al. (2014) Biotechnol. Bioeng. 111(1):25-36), and Saccharomyces cerevisiae (Hodgman & Jewett (2013) Biotechnol. Bioeng. 110(10):2643-54; Gan & Jewett (2014), supra). A plant-based, cell-free expression system is also described in U.S. Pat. No. 10,612,031.

However, uses of the foregoing cell-free systems are generally unsuitable or less informative about gene expression in commercially important crop plants: soybean and maize. Soybean (Glycine max) and maize (Zea mays or corn) are the most planted transgenic crop seeds in the world. Soybeans are the world's largest source of animal protein feed and the second largest source of vegetable oil. It is also used in food products such as soy sauce and tofu. Genetically modified soybeans are found on 94% of acres planted in the United States, 97% of acres in Brazil, 83% of acres in Canada, and from 78-82% of soybean acres worldwide according to information from the United States Department of Agriculture (“USDA”) and International Service for the Acquisition of Agri-biotech Applications (“ISAAA”). Maize is the largest source of feed grain in the United States and is the most important source of grain worldwide, based on production levels. It is also used to produce corn sweeteners and ethanol for fuel. Global maize production levels have exceeded more than 1 billion metric tons per year in recent years. Genetically modified maize is found on 92% of acres in the United States and has been extensively adopted in South American maize-producing countries such as Brazil and Argentina.

To evaluate transgene expression of target gene products in soybean or maize, testing is currently performed in transgenic plant tissue (in planta or ex planta). Such plant tissue can be generated by stable or transient transformation methods. While stable transformed plant is more likely to provide reliable, long-term, and experimentally repeatable information about transgene expression, stable transformation methods can be extremely time consuming, expensive, and subject to very low efficiency rates. Projects involving stable transformation can require screening hundreds or thousands of plants to generate and identify suitable, stably transgenic plants. Transiently transformed plant tissues, by contrast, can be generated more quickly, sometimes in as little time as a few days. However, it can be difficult to interpret results of transient transgene expression, e.g., because it may be difficult or impractical to determine the number of transgenes incorporated in different tissues or different tissue samples, which can make it hard to know if differences in transgene expression are due to differences in the (i) transgene construct, (ii) the expressed transgene product or (iii) different transgene copy numbers in different tissues or tissue samples.

Therefore, there is a desire for soybean and maize based cell-free expression systems.

### BRIEF SUMMARY OF THE DISCLOSURE

Provided herein are systems and methods based on the discovery and development of soybean and maize cell-free, lysate-based reaction systems for the in vitro synthesis of biopolymers (e.g., polynucleotides, polypeptides, polysaccharides, and complex carbohydrates). As compared to transgenic plant or tissue based systems—which can take months or longer to execute, the disclosed systems and methods provide a much quicker, comparatively simpler, and economical way of screening templates (e.g., transgene coding sequences and transgene expression constructs) intended to express biopolymers (e.g., encoded RNA and/or protein products) in soybean or maize. The disclosed cell free protein expression systems (CFPS) based on soybean or maize cell-free lysates also can be used for the expression of biopolymers such as polynucleotides, polypeptides, polysaccharides, and complex carbohydrates, e.g., when other expression systems are incapable, unsuited, or undesirable for the production of a biopolymer.

Described herein is a method for synthesis of a biopolymer that comprises combining a soybean or maize cell-free lysate with a biopolymer template, and monomeric units of the biopolymer in a reaction volume. The biopolymer template can be an RNA molecule, which provides template to produce a polypeptide (a biopolymer) from amino acids (monomers). Reactions using DNA as the biopolymer template may be utilized to produce further nucleic acid molecules (e.g., DNA and RNA) as a biopolymer from monomeric nucleotides through in vitro replication or transcription reaction. Reactions using RNA as the biopolymer template may be used to produce polypeptides in an uncoupled translation reaction. Reactions using DNA as the biopolymer template may also be used to produce polypeptides through a coupled transcription-translation reaction. Thus, disclosed herein is a method for synthesis of a biopolymer comprises combining a soybean or maize cellular lysate with a DNA or RNA template, and monomeric units (e.g., nucleic acids and/or amino acids) of the biopolymer in a reaction volume. In particular examples, the reaction volume does not comprise added creatine phosphate/creatine kinase energy regeneration system, and no phosphate or minimal phosphate needs to be added during the biopolymer synthesis reaction.

In one aspect, described herein is a method of growing soybean cell culture suitable for lysis and use in the disclosed cell-free synthesis of a biopolymer, e.g., a protein. For example, soybean cells can be grown in media containing salts, sugar, auxin (with or without cytokinin), and vitamins. Examples of media are shown in Table 1, below. In Table 1, Gamborg refers to Gamborg B5 basal salt mixture+vitamins, which can be used at concentrations of 2-5 g/L, e.g., 2, 3, 4, or 5 g/L of media. In Table 1, MS refers to media containing Murashige+Skoog salts, which can be used at concentrations of 3-6 g/L, e.g., 4-5, 4.3, 4.4, or 4.5 g/L. In Table 1, sucrose can be used at concentrations of 25-35 g/L, e.g., 25, 30, or 35 g/L. In Table 1, auxins can be 2,4-Dichlorophenoxyacetic acid (2,4-D) which can be used at concentrations of 0.2-1.5 mg/L, e.g., 0.2, 0.4, 0.05 or 1 mg/L, and auxins can also include 1-Naphthaleneacetic acid (NAA) which can be used at concentrations of 0.2 to 1 mg/L, e.g., 0.2, 0.4-0.6 or 0.5 mg/L. In Table 1, a cytokinin can be BAP used at concentrations of 0.02-0.2 mg/L, e.g., 0.02, 0.04, 0.1, or 0.2 mg/L. Some media can be supplemented with inositol source, such as myo-Inositol, at 5-500 mg/L, e.g., 50, 100, or 200 mg/L with or without thiamine HCl supplementation at 1-500 mg/L, e.g., 1, 50, 100, or 200 mg/L. Additionally, each of the media shown in Table 1 can be supplemented with an anti-foaming agent, such as Pluronic surfactant (e.g., CAS number 9003-11-6, which is Poly(ethylene glycol)-block-poly(propylene glycol)-block-poly(ethylene glycol) or PEG-PPG-PEG, average Mn˜2,000, available from BASF (Mount Olive, NJ USA) in solution as Pluronic L-61), to achieve a final surfactant (PEG-PPG-PEG) concentration of 0.0001 to 0.0010% (w/v), e.g., 0.0003% at 0.0004%, 0.0005%, 0.0006%, 0.0007% (w/v). Anti-foaming agents can be used in larger fermentation volumes. Such a medium, as described for Table 1, can be inoculated with soybean cells, which are grown to a target cell density for lysate production. Target cell density for soybean lysate can be packed cell volume (PCV) of 15-30% or 20-25% PCV.

TABLE 1

| | | | | | | Supplement | Supplement |
|---|---|---|---|---|---|---|---|
| | Salt | Sugar | Auxin | Cytokinin | Buffer | 1 | 2 |
| 1 | Gamborg | Sucrose | 2, 4-D, | BAP | | | |
| | | | NAA | | | | |
| 2 | MS | Sucrose | 2, 4-D | — | Potassium | Inositol | Thiamine |
| | | | | | Phosphate | | |
| 3 | MS | Sucrose | 2, 4-D | BAP | Potassium | Inositol | Thiamine |
| | | | | | Phosphate | | |

Also provided is a (i) soybean cell-free lysate that can be used for biopolymer synthesis according to any of the methods disclosed herein and (ii) method for making such a lysate by isolating and lysing soybean protoplasts. In particular, preferred agents for use in density gradients used to generate soybean protoplasts are provided. For example, soybean cells can be cultured to a target density in growth media, then isolated from growth media and resuspended in a protoplast isolation solution that includes appropriate osmotic agent and membrane stabilizer. The cell walls of the resuspended cells can be degraded, e.g., by mechanical or enzymatic treatment to create protoplasts, and the treated cells (protoplasts) can be separated from vacuoles by density gradient centrifugation, e.g., using density gradient media comprising Percoll in evacuolation buffer. Such a gradient can comprise 3-6 density layers that range from 0% to as high as 70% (v/v) of the density gradient media. In one example, such a density gradient comprises layers of 0, 15, 30, 40, and 50% (v/v) of the density gradient media. Evacuolated protoplasts (miniprotoplasts) are enriched in and recovered from one or more layers in the density gradient, in which miniprotoplast layers are physically separated from the vacuole-containing layers. For example, soybean miniprotoplasts can be recovered from one or more layers including the 40% Percoll density gradient layer, one or more layers including the 50% Percoll density gradient layer, or a portion of the density gradient comprising the interface of the 40% and 50% Percoll layers. Soybean miniprotoplasts can then be washed, lysed (e.g., by homogenizer), and separated from large membranes, to prepare a soybean lysate suitable for use in any cell-free biopolymer synthesis method disclosed herein. Additionally, soybean lysate prepared according to the disclosure can be packaged in a kit for synthesis of a biopolymer.

In another aspect, disclosed herein is a method of growing a maize cell culture suitable for lysing and use in cell-free synthesis of a biopolymer, e.g., a protein. In one aspect, maize cells are grown in media containing salts, sugar, auxin (with or without cytokinin), and vitamins. Examples of maize cell culture media can include MS (Murashige+Skoog) salts at concentrations of 3-6 g/L, e.g., 4-5, 4.2, 4.3, 4.4, or 4.5 g/L; sucrose can be used at concentrations of 25-35 g/L, e.g., 25, 30, or 35 g/L; auxins such as 2,4-Dichlorophenoxyacetic acid (2,4-D) can be used at concentrations of 0.2-1.5 mg/L, e.g., 0.2, 0.4, 0.5 or 1 mg/L; Gamborg B5 basal salt mixture+vitamins, which can be used at concentrations of 0.01 to 5 g/L, e.g., 0.05, 0.1, 0.2, 0.3, 0.4 g/L, 0.5, or 1 g/L.

Also provided is (i) a maize cell-free lysate that can be used for biopolymer synthesis according to any of the methods disclosed herein and (ii) a method for making such a lysate by isolating and lysing maize protoplasts. In particular, preferred agents for use in density gradients used to generate maize protoplasts are provided. For example, maize cells can be cultured to a target density in growth media, then isolated from growth media and resuspended in a protoplast isolation solution that includes appropriate osmotic agent and membrane stabilizer. The resuspended cells' cell walls can be degraded, e.g., by mechanical or enzymatic treatment to create protoplasts, and the treated cells (protoplasts) can be separated from vacuoles by density gradient centrifugation, e.g., in a Percoll, sucrose, or iohexol (trade name Nycodenz®) density gradient. Such a gradient can comprise 3-6 density layers that range from 0% to as high as 60% (v/v) of the density gradient media. In one example, such a density gradient comprises layers of 0, 5, 10, 15, 30, 40, and 50% (v/v) of Percoll or sucrose gradient. In another example, such a density gradient comprises from 0% to as high as 30% iohexol layers. Preferably such a density gradient comprises layers of 0, 5, 10, 15, and 20% iohexol (Nycodenz®). Evacuolated protoplasts (miniprotoplasts) are enriched in and recovered from one or more layers (which are physically separate from vacuole-containing layers), e.g., one or more layers including the 10% Nycodenz® layer, one or more layers including the 15% 10% Nycodenz® layer, or a portion of the density gradient comprising the interface of the 10% and 15% Nycodenz® layers. Maize miniprotoplasts can then be washed, lysed (e.g., by homogenizer), and separated from large membranes, to prepare a maize lysate suitable for use in any cell-free biopolymer synthesis method disclosed herein. Additionally, a maize lysate prepared according to the disclosure can be packaged in a kit for synthesis of a biopolymer.

The disclosed cellular lysate systems may be supplemented with only a minimal amount of exogenous creatine phosphate, a minimal amount of creatine kinase or both, so long as the reaction volume comprises exogenous creatine phosphate and/or creatine kinase in amounts considered unsuitable for an energy regeneration system. For example, a reaction volume may contain no more than 15 mM, no more than 10 mM, no more than 5 mM, no more than 1 mM, no more than 500 μM, no more than 100 μM, no more than 50 μM, or no more than 10 μM added creatine phosphate. In another example, a reaction volume may contain no more than 100 μg/mL, no more than 50 μg/mL, no more than 10 μg/mL, no more than 5 μg/mL, no more than 1 μg/mL, than 0.5 μg/mL, or no more than 0.1 μg/mL added creatine kinase. These amounts are unsuitable for, and thus require the inclusion of cellular organelle such as plastids, mitochondria or chloroplasts in accordance with the methods and systems disclosed herein, to sustain biopolymer synthesis (including to sustain biopolymer synthesis for the prolonged periods disclosed herein). Thus, in particular examples, the reaction volume comprises a soybean or maize cellular lysate with a biopolymer template, and monomeric units of the biopolymer in a reaction volume that comprises: (1) no more than 15 mM added creatine phosphate (also referred to as phosphocreatine or PCr) and no more than 50 μg/mL, no more than 10 μg/mL, no more than 5 μg/mL, no more than 1 μg/mL, no more than 0.5 μg/mL, or no more than 0.1 μg/mL added creatine kinase (CK); (2) no more than 10 mM added PCr and no more than no more than 50 μg/mL, no more than 10 μg/mL, no more than 5 μg/mL, no more than 1 μg/mL, no more than 0.5 μg/mL, or no more than 0.1 μg/mL added CK; (3) no more than 5 mM added PCr and no more than no more than 50 μg/mL, no more than 10 μg/mL, no more than 5 μg/mL, no more than 1 μg/mL, no more than 0.5 μg/mL, or no more than 0.1 μg/mL added CK; (4) no more than 1 mM added PCr and no more than no more than 50 μg/mL, no more than 10 μg/mL, no more than 5 μg/mL, no more than 1 μg/mL, no more than 0.5 μg/mL, or no more than 0.1 μg/mL added CK; (5) no more than 500 μM added PCr and no more than no more than 50 μg/mL, no more than 10 μg/mL, no more than 5 μg/mL, no more than 1 μg/mL, no more than 0.5 μg/mL, or no more than 0.1 μg/mL added CK; (6) no more than 100 μM added PCr and no more than no more than 50 μg/mL, no more than 10 μg/mL, no more than 5 μg/mL, no more than 1 μg/mL, no more than 0.5 μg/mL, or no more than 0.1 μg/mL added CK The reaction volume can comprise no more than 10 mM, no more than 5 mM, no more than 1 mM; (7) no more than 50 μM added PCr and no more than no more than 50 μg/mL, no more than 10 μg/mL, no more than 5 μg/mL, no more than 1 μg/mL, no more than 0.5 μg/mL, or no more than 0.1 μg/mL added CK; (8) no more than 10 μM added PCr and no more than no more than 50 μg/mL, no more than 10 μg/mL, no more than 5 μg/mL, no more than 1 μg/mL, no more than 0.5 μg/mL, or no more than 0.1 μg/mL added CK.

Also disclosed are systems for synthesis of a biopolymer without using an artificial energy regeneration system, i.e., no added creatine phosphate and no added creatine kinase. The reaction volume of such a system comprises an aqueous soybean or maize cellular lysate, a (endogenous or heterologous) cellular organelle, a biopolymer template and includes monomeric units of the biopolymer; and the reaction volume does not comprise added creatine phosphate/creatine kinase energy regeneration system, and no phosphate or minimal phosphate needs to be added during the biopolymer synthesis reaction.

In another aspect, the reaction volume does not require amino acid supplementation to support polypeptide synthesis when amino acids present in the soybean or maize lysate disclosed herein may be sufficient to support extended synthesis of polypeptides.

Disclosed herein is a kit for synthesis of a biopolymer that comprises a soybean or maize cellular lysate prepared as disclosed herein. The kit may further comprise one or more of the following: (i) a buffer for in vitro transcription or translation to generate a biopolymer, (ii) monomeric units of the biopolymer, (iii) and/or, optionally a vector or construct into which a template encoding the biopolymer may be inserted (e.g., by molecular genetic techniques). The foregoing kit component(s) may be dispensed in separate volume containers, together with instructions specifying the admixture of the kit components and any exogenous components without creatine phosphate and creatine kinase. By way of further example, a kit may further comprise one or more components such as magnesium, potassium, nucleosides, enzymes (e.g., RNA polymerase), and chloramphenicol. Each component of the kit may be disposed in a separate individual volume that is either ready-to-use or concentrated (e.g., 5× or 10×, so that it requires dilution prior to use). A kit for synthesis of a biopolymer according to specific embodiments may comprise a cellular lysate disclosed herein (e.g., comprising chloroplasts and/or mitochondria), monomeric units of a biopolymer (e.g., nucleosides or amino acids), and written instructions. In such specific embodiments, the written instructions may direct a user to combine these components with a biopolymer template of interest (e.g., a DNA or RNA molecule encoding a polypeptide) and any other reagents, without adding creatine phosphate and/or without adding creatine kinase (for energy regeneration) to the combination.

Embodiments of the methods, systems, and kits herein incorporate active mitochondria for energy regeneration in an ongoing synthesis reaction, and thereby may be utilized to quantitatively investigate compounds or proteins affecting mitochondrial function within the context of in vitro synthesis. Furthermore, intermediates of the TCA cycle may be utilized during the synthesis reaction to produce amino acids, such that amino acid supplementation is not required for prolonged polypeptide synthesis. In some embodiments, a soybean or maize cellular lysate for use in the methods, systems, and kits herein comprises chloroplasts, which reduces oxygen-dependency of the synthesis reaction. For example, a cellular lysate may be prepared from photosynthetic active cells, such that plastids, chloroplasts and/or mitochondria are retained in the lysate, while undesirable cellular material is removed. In such specific examples, the methods, systems, and kits herein may utilize plastid-derived energy, mitochondrion-derived energy regeneration, chloroplast-derived energy regeneration, or a combination of both.

In one aspect, the disclosed soybean lysate and related methods, systems, and kits disclosed herein can be used to synthesize a biopolymer (e.g., polypeptide) for extended periods that provide increasing yields of synthesized biopolymer, including extended periods of 1 hour or more, 2 hours or more, 3, hours or more, 4 hours or more, 5 hours or more, 6 hours or more, or 7 hours or more, 8 hours or more, 9 hours or more, 10 hours or more, 11 hours or more, 12 hours or more, 13 hours or more, 14 hours or more, 15 hours or more, 16 hours or more, 17 hours or more, 18 hours or more, 19 hours or more, 21 hours or more, 22 hours or more, 23 hours or more or 24 hours or more. Additionally, the disclosed soybean lysate and related methods, systems, and kits disclosed herein can be used to synthesize polypeptide yields of 25 micrograms per mL (μg/mL), 50 μg/mL, 75 μg/mL, 100 μg/mL, 125 μg/mL, 150 μg/mL, 175 μg/mL, 200 μg/mL, 225 μg/mL, or 500 μg/mL of synthesis reaction volume.

In another aspects, the disclosed maize lysate and related methods, systems, and kits disclosed herein can be used to synthesize a biopolymer (e.g., polypeptide) for extended periods that provide increasing yields of synthesized biopolymer, including extended periods of 1 hour or more, 1.5 hours or more, 2 hours or more, 2.5 hours or more, 3 hours or more, 3.5 hours or more, 4 hours or more, 4.5 hours or more, or 5 hours or more. Further the disclosed maize lysate and related methods, systems, and kits disclosed herein can be used to synthesize polypeptide yields of 1 micrograms per mL (μg/mL), 1.5 μg/mL, 2 μg/mL, 2.5 μg/mL, 3 μg/mL, 3.5 μg/mL, 4 μg/mL, 4.5 μg/mL, 5 μg/mL, or 5.5 μg/mL of synthesis reaction volume.

The foregoing and other features will become more apparent from the following detailed description of several embodiments, which proceeds with reference to the accompanying figures.

### BRIEF DESCRIPTION OF THE FIGURES

FIG. 1 is a graph that shows productivity (yield) of soybean cell-free lysate for biosynthesis of a biopolymer (protein) at different reaction times. Soybean cellular lysates were prepared as described herein from evacuolated protoplasts isolated in the (a) 40/50% interface of a Percoll density gradient or (b) 50/70% interface of a Percoll density gradient. Yield (micrograms of protein per mL of reaction mix) data at each time point (hours) represents the average (with standard deviation bars) of three independent translation reactions that produced enhanced yellow-fluorescent protein (eYFP).

FIG. 2 is a histogram that shows the relative productivity of maize cell-free lysate for biosynthesis of a biopolymer (eYFP) when encoded by different indicated expression constructs. Maize cell free lysates were prepared from evacuolated protoplasts isolated in the 30/40% interface of a Percoll density gradient. Yield (micrograms of protein per mL of reaction mix) data represents the average (with standard deviation bars) of three independent translation experiments.

### DETAILED DESCRIPTION

### I. Overview of Several Embodiments

The disclosed cell-free expression synthesis (CFPS) systems based on crude soybean and corn lysates provide several advantages over in vivo systems, and are useful in a broad range of applications, including, inter alia, protein engineering, bio-pharmaceutical production, and research. For example, the production of biopolymers (e.g., proteins) intended for delivery to humans or animals can be required to meet safety standards. When biopolymers intended for human or animal administration or consumption are produced using cells that include toxins or using cell lysates derived from organisms include toxins or undesirable factors, it may be necessary to devise, validate and perform rigorous purifications protocols to ensure their safety. The need for rigorous purification can be difficult and can add, time, costs, and uncertainty to the production of biopolymers. Purification can result in the loss or waste of biopolymer, and thereby lower yield. Therefore, it may be preferable to use cell sources and lysates that are generally recognized as safe (GRAS). Soybean and corn are generally recognized as safe.

In another example, the disclosed soybean or maize CFPS provide a faster, cheaper, and simpler way of testing candidate genes and/or transgene constructs, relative to current methods of testing such candidates in transgenic plant tissue (in planta or ex planta). The methods and compositions disclosed herein mimic the cytoplasmic environment of a soybean or maize cell, and result in protein production and protein folding that can be predictive of protein production and protein folding in a soybean or maize plant, respectively. Thus, the disclosed soybean or corn lysates can be used to screen and evaluate the transcription and/or translation efficiency of candidate genes and/or transgene constructs. The disclosed methods and soybean or corn lysates can be used to screen and evaluate different isoforms or mutants of a gene or gene product. Thus the disclosed lysates and CFPS can be used to rapidly and more easily screen for transcription, translation, proper folding, predicted activity, and the like among (i) different coding sequences and/or (ii) different proteins (e.g., a panel of mutants) produced using the soybean or maize lysate transcription and/or translation apparatus. And this can be done without having to transforming, screen and selecting transgenic plants or transgenic plant tissue.

Because the disclosed soybean and maize cell-free lysates contain necessary components for translation, protein folding, and energy metabolism, almost any protein encoded by a RNA template to be synthesized therein in the presence of amino acids, nucleotides, and salts, provided that the lysate is supplemented with energy-storing reagents. In coupled transcription/translation systems, an RNA polymerase can be added to a disclosed soybean or maize cell-free lysates direct the synthesis of a protein from a DNA template. In contrast to cellular synthesis, CFPS may allow shorter process times, reduced protein hydrolysis, and the ability to express toxic proteins or proteins containing specific chemical groups or unnatural amino acids at defined positions. Furthermore, the reaction may be controlled and monitored directly.

The disclosed soybean or maize CFPS can also be used to generate biopolymers that cannot be generated inplanta or explanta such as nucleic acids that include non-canonical, non-naturally occurring, modified, labeled or toxic monomers or substrates, e.g., (i) nucleoside analogs, (ii) toxic amino acids (e.g., canavanine, hydroxylysine, or d-amino acids), or (iii) radiolabeled or fluorescent versions of the monomer to be incorporated into a biopolymer. See, e.g., Cui et al. (2020), Frontiers Bioengineering and Biotechnology, 8: Article 1031. The foregoing can be used to expand the functionality of a biopolymer produced using the disclosed CFPS.

Some conventional eukaryotic cell-free systems (e.g., wheat germ extract, and insect cell extract) lack mitochondria. Instead, these systems require the addition of creatine phosphate and creatine kinase to accomplish the necessary ATP regeneration to support protein expression. The large accumulation of free phosphate (derived from the creatine phosphate) that is added to the reaction mixture for energy regeneration in order to support protein expression is a significant limiting factor in the performance of these systems. Ezure et al. (2006) Biotechnol. Prog. 22(6):1570-7; Takai et al. (2010) Curr. Pharm. Biotechnol. 11:272-8; Brödel et al. (2014) Biotechnol. Bioeng. 111(1):25-36; Hodgman & Jewett (2013), supra; Schoborg et al. (2014) Biotechnol. J. 9(5):630-40. The free phosphate introduced into the system binds magnesium (which is needed for transcription and translation), resulting in an early breakdown of the synthetic performance and low product yields.

In order to prolong the synthetic performance, some currently available eukaryotic cell-free systems use “continuous flow” reactions in dialysis mode to provide a long-lasting energy supply and to dilute inhibitory components like phosphate in the reaction compartment. Systems without an artificial energy regeneration system for biopolymer synthesis described herein can reduce or eliminate the need for reaction dialysis, as they produce less inhibitory components, and have their own energy regenerative capacity. However, the systems herein may be utilized in a continuous flow configuration if desired, according to the discretion of the practitioner.

The systems herein offer the possibility to investigate compounds or pathways affecting mitochondrial and/or chloroplast function, for example, as further enhancers of cell-free protein expression. Thus, the fundamentally different systems of embodiments herein may be optimized to provide even further benefits.

Some systems for biopolymer synthesis described herein are capable of supporting growth of microorganisms, and such growth may result in depletion of substrates in CFPS reaction and/or protein degradation, resulting in reduced yield of a target protein. Therefore, in some embodiments, the system includes chloramphenicol to inhibit microbial growth, which may improve protein expression in these embodiments. In particular embodiments, the system includes chloramphenicol in an amount between, for example, 10-500 μg/mL (e.g., between 25-250 μg/mL, between 50-200 μg/mL, and between 100-200 μg/mL).

In a coupled in vitro transcription/translation (IVTT) reaction, the disclosed soybean or maize CFPS system may include approximately 0.1-10 mM ATP (e.g., 0.1 or 0.5, 1 or 2 or 3, or 4 or 5, or 6, or 7 or 8 or 9 or 10 mM ATP), approximately 0.1-10 mM GTP (e.g., 0.1 or 0.5, 1 or 2 or 3, or 4 or 5, or 6, or 7 or 8 or 9 or 10 mM GTP), approximately 0.1-10 mM CTP (e.g., 0.1 or 0.5, 1 or 2 or 3, or 4 or 5, or 6, or 7 or 8 or 9 or 10 mM CTP), and approximately 0.1-10 mM UTP (e.g., 0.1 or 0.5, 1 or 2 or 3, or 4 or 5, or 6, or 7 or 8 or 9 or 10 mM UTP). A further advantage of utilizing these reduced amounts of NTPs is reduced expense, as the amount of GTP (the most expensive NTP) is reduced from that of a conventional plant cell system.

In certain examples addition glucosylglycerol can be used in an IVTT reaction. For example, glucosylglycerol can be added to maize or soybean lysate disclosed herein in amounts between 0.25% and 4%. Without being bound to any particular theory, glucosylglycerol may improve reaction yield by increasing protein and membrane stability. Therefore, in some embodiments, the system includes 0.25-4% (e.g., 0.25-2%, 0.25-1%, about 0.5%, and 1.5%) glucosylglycerol. By way of further example, even though it is not required, the addition of branched amino acids (BCAAs) BCAAs can be added to maize or soybean lysate disclosed. Therefore, in some examples, the maize or soybean lysate disclosed system is supplemented with BCAAs in amounts between about 0.25-4 mM or in amounts from 0.5-2 mM (e.g., 0.48-2.2 mM, 0.5-2.0 mM, 0.5-1 mM, and about 1 mM).

The disclosed soybean lysate-based CFPS can be used to produce increasing amounts of a target protein in for more than 20 hours. In one example, reactions produce about 250 μg/mL eYFP.

### II. Abbreviations

AAD-12 aryloxyalkanoate dioxygenase-12
ADP adenosine diphosphate
ATP adenosine triphosphate
BCAA branched chain amino acid
CFPS cell-free expression synthesis
CL cellulase enzyme
CK creatine kinase
CP creatine phosphate
Cry Bacillus thuringiensis Cry proteins (endotoxins)
CTP cytidine triphosphate
DMSO dimethyl sulfoxide
DTT dithiothreitol
EDTA ethylenediaminetetraacetic acid
eYFP enhanced yellow fluorescent protein
GTP guanosine triphosphate
IVTT in vitro transcription and translation
NTP nucleoside triphosphate
PCR polymerase chain reaction
PEG polyethylene glycol
TCA tricarboxylic acid cycle (“Krebs cycle”)
UTP uridine triphosphate
UTR untranslated region

### III. Terms

Exogenous: The term “exogenous,” as applied to components (e.g., plastids, mitochondria or chloroplasts) added to cellular lysate herein, refers to such components having a different origin than the cell lysate. For example, the term exogenous is used herein to refer to a template (e.g., a nucleic acid molecule) that is added to lysate and that directs the lysate to synthesize a biopolymer (e.g., a polypeptide). In one aspect an exogenous template refers to a nucleic acid molecule comprises a sequence that is not found in the soybean or maize cells from which lysate was generated (e.g., transgenic coding sequence or regulatory sequence from a different species, a codon-optimized sequence, an artificial sequence that has been modified or designed by the user). An exogenous template can refer to sequence encoding a transgene, gene suppressing RNA (RNAi, microRNA, dsRNA, siRNA, antisense polynucleotide, RNA induced silencing complex), gene editing effectors (e.g., ZFN, CRISPR, TALEN components), or therapeutic RNA or polypeptide molecules. The term exogenous can also refer to plastids, mitochondria or chloroplasts, which do not originate from the cellular lysate and which are added to the lysate, are exogenous to the cellular lysate. The term exogenous, may be applied to cell organelles such as plastids, mitochondria or chloroplasts from the same cell type or from a different cell type (e.g., cell from a different tissue or different species) as the cell type used to derive the cellular lysate, so long as in either case the organelles are not derived from the cellular lysate itself. Additionally, the term exogenous may be used herein to refer to components of an energy regeneration system (e.g., creatine phosphate and creatine kinase) that are added separately or in addition to any plastid, mitochondria or chloroplast organelles used in the cell lysate of a system disclosed herein.

Isolated: An “isolated” biological component (such as a nucleic acid or protein) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component (e.g., a nucleic acid may be isolated from a chromosome by breaking chemical bonds connecting the nucleic acid to the remaining DNA in the chromosome). Nucleic acid molecules and proteins that have been “isolated” include nucleic acid molecules and proteins purified by standard purification methods. The term also embraces nucleic acids and proteins prepared by recombinant expression in a host cell, as well as chemically-synthesized nucleic acid molecules, proteins, and peptides.

Nucleic acid molecule: As used herein, the term “nucleic acid molecule” may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A nucleic acid molecule can include either or both naturally occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, and carbamates; charged linkages: for example, phosphorothioates, and phosphorodithioates; pendent moieties: for example, peptides; intercalators: for example, acridine, and psoralen; chelators; alkylators; and modified linkages: for example, and alpha anomeric nucleic acids). The term “nucleic acid molecule” also includes any topological conformation, including single-stranded, double-stranded, partially duplexed, triplexed, hairpinned, circular, and padlocked conformations.

As used herein, “about” used before a numerical value(s) means a value between 90% and 110% of the recited numerical value(s).

### IV. Systems for Biopolymer Synthesis

This disclosure provides systems for synthesis of a biopolymer that use soybean or maize cell lysates. The system may be a combination of CFPS reaction components, which may be assembled and mixed by the practitioner, or which may be provided to the practitioner in a kit as premixed components, unmixed components, or a combination of the two. The practitioner may utilize some or all of the components of a kit in combination with a DNA or RNA template, which is provided by the practitioner to synthesize a protein chosen by the practitioner. Once all the components of the system are mixed in a reaction volume under appropriate environmental conditions, the reaction commences, and proceeds generally according to conventional in vitro cell-free synthesis reactions, with important changes described herein. The reaction may be allowed to proceed until one or more of the components (e.g., NTPs and amino acids) are exhausted in the reaction volume, or until it is halted by adjusting the environmental conditions to end the energy regeneration process in the system.

Systems of embodiments herein are useful for the production/replication of biopolymers, including, for example, amplification of DNA, transcription of RNA from DNA or RNA templates, translation of RNA into polypeptides, and the synthesis of complex carbohydrates from simple sugars. Enhanced synthesis includes in some examples one or more of: increases in the total or relative amount of biopolymer synthesized in the system; increases in the total or relative amount of biopolymer synthesized per unit of time; increases in the total or relative amount of biologically active biopolymer (e.g., properly folded and/or post-translationally modified protein) synthesized; increases in the total or relative amount of soluble biopolymer synthesized, and reduced expense in time and/or money required to synthesize a given amount of biopolymer.

Particular embodiments herein accomplish the translation of mRNA to produce polypeptides, which translation may be coupled to in vitro synthesis of mRNA from a DNA template. Such a cell-free system contains all the factors required for the translation of mRNA, for example, ribosomes, amino acids, tRNAs, aminoacyl synthetases, elongation factors, initiation factors, and ribosome recycling factors. In examples herein, such a cell-free system comprises a cell lysate prepared in the manner described herein from a soybean or maize cell.

### Cell Lysate

The disclosed cell-free lysates retain a variety of post-translational processing activities that can be used or adapted to support the in vitro translation of a wide variety of viral and other prokaryotic RNAs, as well as eukaryotic mRNAs. Template mRNAs that have a codon usage that deviates from that of soybean or maize, may still be used in the disclosed cell lysates, for example, by supplementing the system with rare tRNAs and/or amino acids in the organism.

Preparation of a cell lysate according to embodiments herein may include, inter alia, disruption/removal of cell walls (for plant cells) and cell membranes, removal of lytic vacuoles, and removal of endogenous mRNAs.

Cell walls and membranes may be disrupted in some embodiments by techniques including, for example and without limitation, mechanical disruption, liquid homogenization, enzymatic digestion, high frequency sound waves, decompression, freeze/thaw cycles, and manual grinding. In particular embodiments herein, plant cell lysates are prepared by digesting the cell wall using one or more cell-wall digesting enzymes (e.g., Cellulase Onozuka RS™ Pectolyase Y-23™, Macerozyme R-10), and liquid enzymes (e.g., Rohament CL™, Rohament PL™, and Rohapect UF™, which were originally intended for the production of fruit juice and extracts). Rohament CL™ comprises a cellulase concentrate, Rohament PL™ is a pectinase concentrate, and Rohapect UF™ contains an enzyme complex including specialized pectinases and arabanases. The use of these enzyme combinations reduced the costs of protoplastation more than 100-fold, as compared to conventional methods.

Also during the lysate preparation, any lytic vacuoles may be removed. Such vacuoles contain undesirable enzymes, including proteases and ribonucleases, which interfere with the synthesis of polypeptides and mRNAs. In some embodiments herein, lytic vacuoles are removed by centrifugation in a Percoll gradient, or any other density gradient. Vacuoles have a low density, and thus can be separated from protoplasts, yielding high-density evacuolated protoplasts. In some examples, a stepwise Sucrose density gradient may be utilized for evacuolation, where the protoplasts are applied directly onto the Percoll-free top layer. After centrifugation, the evacuolated protoplasts will be separated from the vacuoles, for example, concentrated at the interface between the appropriate gradient layers, whereas the separated, lower-density vacuoles will be in a lower-density gradient; for example, floating on the top layer.

Evacuolated protoplasts may then be washed and then disrupted by a Dounce tissue grinder or nitrogen decompression to protect labile cell components from oxidation. After the removal of nuclei and non-disrupted cells, the lysate may be treated to destroy endogenous mRNAs while leaving the integrity of the 18S and 28S ribosomal RNAs mainly unaffected, thereby minimizing background translation. In particular examples herein, nuclease S7 is used.

### Template

To direct the synthesis of a biopolymer in the systems herein, a template must be present in the reaction, as stored information to be converted into the polymer. The template for cell-free protein synthesis can be either mRNA or DNA, encoding for any polynucleotide (DNA) or polypeptide (DNA and/or mRNA) of interest. A coupled transcription/translation system continuously generates mRNA from a DNA template with a recognizable promoter. Either the endogenous RNA polymerase may be used, or an exogenous RNA polymerase (e.g., a phage RNA polymerase, typically T7 or SP6), may be added directly to the reaction mixture. Alternatively, mRNA may be continually amplified by inserting the message into a template for QB replicase, an RNA-dependent RNA polymerase. In some embodiments, a vector containing a poly-A sequence at one end of the multiple cloning region is used as a template in an IVTT reaction. For example, such a vector may contain an SP6, T7, or T3 RNA polymerase promoter at the opposite end of the multiple cloning region, so that cloning into the vector produces a gene that is flanked by an RNA polymerase promoter at the 5′ end and a poly-A sequence at the 3′ end. In embodiments wherein mRNA is utilized as the template, the purified mRNA may be stabilized by chemical modification before it is added to the reaction mixture.

The nucleotide sequence of a DNA or mRNA sequence utilized as a template according to embodiments herein may be optimized to achieve higher levels of expression. Several mRNA structural characteristics affect translation efficiency, including untranslated regions (UTRs) at the 5′ and 3′ ends of the coding sequence. The structure of the 5′ UTR influences translational initiation, termination, and mRNA stability. One of the rate-limiting steps in translational initiation is the binding of the mRNA to the 43S pre-initiation complex. The translational machinery is recruited by the 5′-cap, or translational enhancers in the leader sequence. In certain embodiments herein, a template mRNA may contain an untranslated region selected from a group comprising the 5′ UTR in pCITE2a (which contains an internal ribosomal entry site (IRES) derived from Encephalomyocarditis virus (EMCV)); sequences from Barley yellow dwarf virus (BYDV) in vector pF3A; the 5′ UTR from a baculovirus polyhedrin gene; a synthetic 3′ UTR including a poly-A sequence; and a 5′-UTR including an ARC-1 sequence element (which is complementary to an internal 18S rRNA segment, and may promote binding to the 40S ribosomal subunit); the Tobacco mosaic virus (TMV) 5′-UTR (omega sequence), which may be improved by adding a GAAAGA upstream of an initial GUA triplet.

In some embodiments, a DNA molecule is used to produce capped mRNA in vitro, for example, in the presence of the cap analog m7G[5′]ppp[5′]G. Non-incorporated nucleotides and cap analogs may be removed by gel filtration, and the purified mRNA may then be introduced into the cell-free system as described herein, where it serves as the template for polypeptide synthesis.

### Monomers

In coupled IVTT reactions, ribonucleotide triphosphates (ATP, GTP, CTP, UTP) and amino acids are required in the system as the monomeric units used to synthesize the desired biopolymers. In some embodiments herein, the system operates with reduced levels of one or more NTPs relative to a comparable system with conventional energy regeneration system. In these embodiments, the disclosed system provides an advantageous reduced expense for the system's operation. In certain embodiments, the disclosed system operates with a final ATP concentration of between 2-10 mM, e.g., 4-8 mM or 5-7 mM ATP. In certain embodiments, the disclosed system operates with a final GTP concentration of between 0.8-2.5 mM, e.g., 1-2 mM or 1.4-1.8 mM GTP. In certain embodiments, the disclosed system operates with a final CTP concentration of between 0.4-2.4 mM, e.g., 0.5-2 mM or 0.6-1.0 mM CTP. Also, in certain embodiments, the disclosed system operates with a final UTP concentration of between 0.4-2.4 mM, e.g., 0.5-2 mM or 0.6-1.0 mM UTP. For example, the system can operate with final NTP concentrations at or about 6 mM ATP, 1.6 mM GTP, 0.8 mM CTP, and 0.8 mM UTP. In particular examples, a synthesis reaction is supplemented with low concentration NTP mix containing approximately 150 mM ATP, approximately 40 mM GTP, approximately 20 mM CTP, and approximately 20 mM UTP and the mix is added to the system in sufficient amount to provide the final concentration of NTPs.

Amino acids may also be added, for example, to a final concentration of 0.05-4 mM. If a radiolabeled amino acid (e.g., ³⁵S methionine and ³H leucine) is used in a coupled reaction, then the corresponding amino acid may be left out of the amino acid mix.

### Salts

The concentration of salts is controlled in systems according to embodiments herein. For example, a system may have added to it one or more salts, including, for example, and without limitation, potassium, magnesium, ammonium, and other biologically relevant salts, such as manganese (e.g., of acetic acid or sulfuric acid). One or more of such salts may have amino acids as a counter anion. There is an interdependence among ionic species with regard to the function of the synthesis reaction. When changing the concentration of a particular ion in the reaction medium, that of another ion may be changed accordingly. For example, the concentrations of added salts may be simultaneously controlled in accordance with the change in other components, such as nucleotides. Furthermore, the concentration levels of components in a continuous-flow reactor may be varied over time.

### Magnesium

Magnesium is important for protein translation, as it enhances ribosome assembly, and the stability of assembled ribosomes. Magnesium also appears to play a role in facilitating polymerase binding. In embodiments herein, the magnesium concentration of the cell lysate may be adjusted by an additional magnesium compound. In some embodiments, the additional magnesium compound is a salt; for example, magnesium chloride, magnesium acetate, and magnesium glutamate. For coupling transcription and translation, a sufficient amount of a magnesium salt may be added to the lysate to raise the final magnesium concentration to a level where RNA is transcribed from DNA, and RNA is translated into protein.

To provide precise control of the magnesium concentration in a system herein, lysate magnesium levels may be measured directly through the use of a magnesium assay, prior to the addition of extra magnesium. The Lancer “Magnesium Rapid Star Diagnostic Kit” (Oxford LabWareDivision™, Sherwood Medical Co., St. Louis, MO), for example, is one assay that can accurately measure the magnesium levels in biological fluid. Once the magnesium ion concentration for a given batch of lysate is known, then additional magnesium may be added to bring the magnesium concentration of the lysate to within the desired range.

As suggested above, the final magnesium concentration in the reaction is affected by other conditions and considerations. Thus, for example, as the ribonucleotide triphosphate concentration goes up, there is a concomitant increase in the optimal magnesium concentration, as the ribonucleotide triphosphates tend to associate, or chelate, with magnesium in solution. Thus, when ribonucleotide triphosphate concentrations are increased, additional magnesium is generally also added to the reaction. The optimal concentration of magnesium also varies with the type of cellular lysate. The amount of magnesium required to be added also varies with the concentration of the lysate used in the reaction mixture, as increasing the concentration of the lysate will increase the contribution of magnesium from the lysate itself.

### Potassium

Potassium is also typically added to the system to achieve desired levels of biopolymer synthesis. Potassium (for example, potassium acetate and potassium glutamate) can be adjusted by varying the amount of potassium included in translation reaction buffer. As is the case for magnesium, the final potassium concentration may vary slightly, due to its presence in endogenous cellular lysate components.

### Additional Components

Additional components may also be added to the system in particular embodiments, as desired for improving the efficiency or stability of the synthesis reaction. For example, metabolic inhibitors to undesirable enzymatic activity may be added to the reaction mixture. Alternatively, enzymes or factors that are responsible for undesirable activity may be removed directly from the extract or the gene encoding the undesirable enzyme may be inactivated or deleted from the chromosome.

Vesicles, either purified from the host organism (See Muller & Blobel (1984) Proc. Natl. Acad. Sci. U.S.A. 81:7421-5), or synthetic, may also be added to the system. These may be used to enhance protein synthesis and folding. For example, the systems described herein also may be used for cell-free reactions to activate membrane proteins; for example, to insert or translocate proteins or to translocate other compounds, and these processes may be aided in particular embodiments by the addition of vesicles containing desired membrane proteins.

In addition to the above components, other materials (such as those specifically utilized in protein synthesis) may be added to a system as described herein. Such materials may include, for example and without limitation, other salts, folinic acid, cyclic AMP, inhibitors of protein or nucleic acid degrading enzymes, RNasin, inhibitors or regulators of protein synthesis, adjusters of oxidation/reduction potential(s) (e.g., redox reagents such as DTT, glutathione and combinations thereof), chloramphenicol, non-denaturing surfactants, buffer components (such as may be used in the solution to stabilize the reaction pH), PEG, Triton X-100, spermine, spermidine, and putrescine.

Some embodiments include a kit including components of a system for synthesis of a biopolymer without using an artificial regeneration system. In particular embodiments, a kit may include a cell lysate. Alternatively, the kit may include cells for culture and expansion to yield cells for the preparation of a cell lysate. In particular embodiments, the kit may include one or more of salts, NTPs, enzymes (e.g., polymerases and nucleases), enzyme inhibitors (e.g., RNasin), template, and other additives (e.g., chloramphenicol). In particular examples, the kit may include a naked vector, into which may be cloned a gene of interest, for use as a template in the system. In kits including a cell lysate, the lysate may be standard, or it may be of the type where the adjustments to its salt concentrations have already been made during manufacture, or additionally where one or more of the components, reagents or buffers necessary for coupled transcription and translation have been included. In particular examples, the kit may not include a template, but instead may rely on the user to provide the template. A kit may comprise a set of instructions, or link to a website comprising instructions, informing the user how to utilize the components of the kit to perform a synthesis reaction.

### V. Methods for Biopolymer Synthesis

The systems as described above may be used in a method for in vitro synthesis of one or more biopolymers. In vitro synthesis refers to the cell-free synthesis of biological macromolecules in a reaction mix comprising biological extracts and/or defined reagents. Using the systems herein, a cell-free synthesis reaction may be performed in batch, continuous flow, and semi-continuous flow configurations, as these configurations are known in the art. In some embodiments, batch-cultured cells may be used. In some embodiments, cells may be grown continuously in a stirred-tank fermenter to ensure a reproducible supply of homogeneous cell material.

There are differences between using a static IVTT reaction, versus a continuous or flow-through reactions, that may be a consideration in some applications, but not others. For example, the continuous system is generally used for large-scale industrial production of proteins, whereas static system reactions are better suited to small scale in vitro translations (e.g., in a research setting). Continuous translation is much more expensive to perform, requiring an investment in equipment, as well as significant amounts of reagents. In particular, the levels of RNA polymerases used to make continuous eukaryotic reactions work may be prohibitive for simple research applications (i.e., as much as 20,000-30,000 U/reaction). Furthermore, continuous reactions are designed to be performed in relatively large volumes, while static reactions require no extra equipment, and only small amounts of reagents, since the reaction volume is typically only on the order of 100 μL or less.

Systems herein may utilize a large scale reactor, a small scale reactor, or may be multiplexed to perform a plurality of simultaneous syntheses. Continuous reactions use a feed mechanism to introduce a flow of reagents, and may isolate the end-product as part of the process. In both continuous and static reactions, additional reagents may be introduced to prolong the period of time for active synthesis. A reactor may be run in any mode such as batch, extended batch, semi-batch, semi-continuous, fed-batch, and continuous, which mode may be selected in accordance with the application purpose.

Reactions may be conducted in any volume, again depending on the application and the equipment used. For example, in a small scale reaction, the reaction volume may be 1-15 μL, at least 15 μL, at least 50 μL, at least 100 μL, at least 0.5 mL, or at least 1 mL, but may be less than 10 mL. In principle, reactions may be conducted at any scale as long as sufficient oxygen (or other electron acceptor) is supplied. For production of the largest amount of product, industrial bioreactors may be used.

Methods herein may utilize a means for isolating the synthesized biopolymer; for example, a protein isolating means. In some embodiments operated in a continuous operation mode, the product output from the reactor flows through a membrane, and into the protein isolating means. In a semi-continuous operation mode, the outside or outer surface of the membrane is put into contact with predetermined solutions that are cyclically changed in a predetermined order. These solutions may contain substrates such as amino acids and nucleotides. At this time, the reactor is operated in dialysis, or diafiltration batch or fed-batch mode. A feed solution may be supplied to the reactor through the same membrane or a separate injection unit. Synthesized protein is accumulated in the reactor, and then is isolated and purified according to the usual method for protein purification after completion of the system operation.

Where there is a flow of reagents, the direction of liquid flow can be perpendicular and/or tangential to a membrane. Tangential flow is effective for recycling ATP, and for preventing membrane plugging and may be superimposed on perpendicular flow. Flow perpendicular to the membrane may be caused or effected by a positive pressure pump or a vacuum suction pump. The solution in contact with the outside surface of the membrane may be cyclically changed, and may be in a steady tangential flow with respect to the membrane. Furthermore, the reactor may be stirred internally or externally by proper agitation means.

During protein synthesis in the reactor, the protein isolating means for selectively isolating the desired protein may include a unit packed with particles coated with antibody molecules or other molecules immobilized with a component for adsorbing the synthesized, desired protein, and a membrane with pores of proper sizes. Preferably, the protein isolating means comprises two columns for alternating use.

The amount of protein produced in a translation reaction can be measured in various fashions. One method relies on the availability of an assay which measures the activity of the particular protein being translated. An example of an assay for measuring protein activity is a luciferase assay system, or chloramphenicol acetyltransferase assay system. These assays measure the amount of functionally active protein produced from the translation reaction. Activity assays will not measure full length protein that is inactive due to improper protein folding or lack of other post-translational modifications necessary for protein activity. Alternatively, specific proteins might be detected according to their size by capillary electrophoresis.

Another method of measuring the amount of protein produced in coupled in vitro transcription and translation reactions is to perform the reactions using a known quantity of radiolabeled amino acid such as 35S-methionine, 3H-leucine, or 14C-leucine, and subsequently measure the amount of radiolabeled amino acid incorporated into the newly translated protein. Incorporation assays will measure the amount of radiolabeled amino acids in all proteins produced in the reaction, including truncated protein products. The radiolabeled protein may be further separated on a protein gel, and by autoradiography confirmed that the product is the proper size, and that secondary protein products have not been produced.

The following are examples of specific aspects of the disclosure. The examples are offered for illustrative purposes only and are not intended to limit the scope of the disclosed inventions in any way.

### EXAMPLES

Example 1: Evaluation of Soybean Plant Material Preparations Soybean (cv Williams-82) cells were cultivated in a shake flask to achieve a packed cell volume of (PCV) 20-25% at 26° C. in the dark based on protocols previously used for tobacco cells (see U.S. Pat. No. 10,612,031). However, the standard tobacco cell protocol resulted in few healthy protoplasts.

Alternative protocols were investigated. The growth media listed in Table 2 were evaluated for protoplast performance by growing cells during a 25-day period that included successive transfer to fresh medium every 3-4 days. Different cultivation media and growth phases of preculture were found to significantly affect ultimate lysate quality.

TABLE 2
Gamborg 1

| | | |
|---|---|---|
| Gamborg B5 salts + Vitamins | 3.16 | g/L |
| Sucrose | 30 | g/L |
| NAA | 500 | μg/L |
| BAP | 500 | μg/L |
| 2,4-D | 500 | μg/L |

pH to 5.7 with 1M KOH/NaOH
Gamborg 2

| | | |
|---|---|---|
| Gamborg B5 salts + Vitamins | 3.16 | g/L |
| Sucrose | 30 | g/L |
| 2,4-D | 200 | μg/L |
| KH2PO4 | 200 | mg/L |
| Thiamine HCl | 1.0 | mg/L |
| Myo-Inositol | 100 | μg/L |

pH to 5.7 with 1M KOH/NaOH
NB Dicamba

| | | |
|---|---|---|
| NB Basal Medium (PhytoTech N492) | 4.1 | g/L |
| L-Dicamba | 6.6 | mg/L |
| Sucrose | 30 | g/L |
| Myo-Inositol | 100 | mg/L |
| Casein-Enzymatic Hydrolase | 300 | mg/L |
| L-Glutamine | 500 | mg/L |
| L-Proline | 4.5 | mM |

pH to 5.8 with 1M KOH/NaOH
MS

| | | |
|---|---|---|
| Murashige + Skoog Salts | 4.3 | g/L |
| Sucrose | 30 | g/L |
| KH2PO4 | 200 | mg/L |
| 2,4-D | 200 | μg/L |
| Thiamine HCl | 1.0 | mg/L |
| Myo-Inositol | 100 | μg/L |

pH to 5.8 with 1M KOH
Modified MS

| | | |
|---|---|---|
| Murashige + Skoog Salts | 4.3 | g/L |
| Sucrose | 20 | g/L |
| KH2PO4 | 250 | mg/L |
| 2,4-D | 200 | μg/L |
| Thiamine HCl | 10 | mg/L |
| Myo-Inositol | 100 | μg/L |
| Nicotinic Acid | 1 | mg/L |
| Pyridoxine | 1 | mg/L |

pH to 5.8 with 1M KOH
Finer and Nagasawa

| | | |
|---|---|---|
| Murashige + Skoog Salts | 4.6 | g/L |
| Sucrose | 30 | g/L |
| KH2PO4 | 200 | mg/L |
| 2,4-D | 200 | μg/L |
| Thiamine HCl | 1 | mg/L |
| Myo-Inositol | 100 | μg/L |

pH to 5.8 with 1M KOH
Schenk & Hildebrandt 1 (S + H 1)

| | | |
|---|---|---|
| Schenk & Hildebrandt salts | 3.2 | g/L |
| Sucrose | 30 | g/L |
| KH2PO4 | 200 | mg/L |
| 2,4-D | 200 | μg/L |
| Thiamine HCl | 1 | mg/L |
| Myo-Inositol | 100 | μg/L |

pH to 5.8 with 1M KOH
Schenk & Hildebrandt 2 (S + H 2)

| | | |
|---|---|---|
| Schenk & Hildebrandt salts | 3.2 | g/L |
| Sucrose | 30 | g/L |
| KH2PO4 | 200 | mg/L |
| 2,4-D | 200 | μg/L |
| Thiamine HCl | 5 | mg/L |
| Myo-Inositol | 1 | g/L |
| Nicotinic Acid | 5 | mg/L |
| Pyridoxine | 0.5 | mg/L |

pH to 5.8 with 1M KOH

In Table 3 the following abbreviations are used: NAA for 1-naphthaleneacetic acid 2,4-D for 2,4-dichlorophenoxyacetic acid, BAP for cytokinin 6-benzylaminopurine. Murashige and Skoog basal salt mixture was from Duchefa™ Biochemie, Haarlem, Netherlands. Thiamine and BAP were stored in water at 4° C. and 2,4-D was stored in dimethyl sulfoxide (DMSO) at −20° C.

The best growth profile and highest protoplast yield among the media listed in Table 2 was achieved using Gamborg 1 media, which includes the following plant hormones: synthetic auxins 1-naphthaleneacetic acid (NAA) and 2,4-dichlorophenoxyacetic acid (2,4-D) and the synthetic cytokinin 6-benzylaminopurine (BAP). Modified MS media was then tested, after modification to include each of these plant hormones as described in Table 3.

TABLE 3
MS A

| | | |
|---|---|---|
| Murashige + Skoog Salts | 4.3 | g/L |
| Sucrose | 30 | g/L |
| KH2PO4 | 200 | mg/L |
| 2,4-D | 1 | mg/L |
| BAP | 0.2 | mg/L |
| Thiamine HCl | 1.0 | mg/L |
| Myo-Inositol | 100 | mg/L |

pH to 5.8 with 1M KOH
MS B

| | | |
|---|---|---|
| Murashige + Skoog Salts | 4.3 | g/L |
| Sucrose | 30 | g/L |
| KH2PO4 | 200 | mg/L |
| 2,4-D | 0.5 | mg/L |
| BAP | 0.1 | mg/L |
| Thiamine HCl | 1.0 | mg/L |
| Myo-Inositol | 100 | mg/L |

pH to 5.8 with 1M KOH
MS C

| | | |
|---|---|---|
| Murashige + Skoog Salts | 4.3 | g/L |
| Sucrose | 30 | g/L |
| KH2PO4 | 200 | mg/L |
| 2,4-D | 200 | μg/L |
| BAP | 40 | μg/L |
| Thiamine HCl | 1.0 | mg/L |
| Myo-Inositol | 100 | mg/L |

pH to 5.8 with 1M KOH

Protoplasts generated by enzymatic treatment were passed through 200 μm pore size mesh and subjected to Percoll gradient centrifugation at 10,000×g for 1 hour. The thickest band of evacuolated protoplasts was obtained from protoplasts derived from cell grown in MS A medium, which performed better than MS B medium, which performed better than Gamborg 1 medium. Significantly higher number of damaged protoplasts was obtained using MS C and original MS media. Therefore, MS A medium was selected for further development.

Following further experiments and testing, the following process was identified to be the best for cultivation of Williams-82 cells and preparing translationally active lysates. Precultures was cultivated in MS_A medium. For cultivation of the main culture, MS_A medium was adapted to MS_A Fermenter (Table 4) that provided improved protoplast formation and subsequent protoplast evacuolation. Relative to MS_A, MS_A Fermenter medium contains less sucrose, more monopotassium phosphate (KH₂PO₄) and the antifoaming agent Pluronic L-61 (BASF™, Mount Olive, NJ, USA).

TABLE 4
MS A Fermenter

| | | |
|---|---|---|
| MS salts | 4.3 | g/L |
| Sucrose | 20 | g/L |
| KH2PO4 | 250 | mg/L |
| Myo inositol | 100 | mg/L |
| Thiamine | 1 | mg/L |
| 2,4-D | 1 | mg/L |
| BAP | 0.2 | mg/L |
| Pluronic L-61 | 0.0005% | (w/v) |

Example 2: Preparation and Scale up for Production of Soy Cell Lysate. Routine soybean cultures were cultivated by adding inoculant to make 5% (v/v) PCV in 50 mL MS_A medium, and growing for 7 days at 26° C. 160 rpm in a 100 mL Erlenmeyer flask with aluminum foil cap. Larger routine culture volumes can be grown in 100 mL culture in 250 mL Erlenmeyer flask.

Preculture was prepared by adding an inoculant of the routine culture cells at a density of 10% (v/v) packed cell volume (PCV) to an Optimum Growth™ 5 L Flask containing 0.4 L of 1 L MS_A medium and was grown at 26° C. and 160 rpm for 3 days. Packed cells of preculture cells was determined by centrifugation of 10 mL preculture cells in 15 mL tube at 500×g for 5 minutes. Main culture for lysate production was started by inoculating 1 L of MS_A Fermenter medium in Optimum Growth™ 5 L Flask with the preculture cells to a final density of 8% (v/v) PCV. The main culture was cultivated at 26° C. and 160 rpm for 3 days to reach a final PCV of 20-25%, which was used for optimized lysate preparation.

Optimized cell-free soybean lysate (SBL) was produced according to the following protocol. (1) Harvest cell suspension into a 2 L beaker and determine the volume of the cell suspension. (2) Weigh sorbitol to final concentration of 0.4 M of the cell suspension then dissolve sorbitol completely in the cell suspension by manual stirring (e.g. with a serological pipette). (3) Add 1 mM CaCl₂) (stabilizes membranes) using a 1 M CaCl₂) stock solution in distilled water and stir manually with a serological pipette until an even distribution is obtained. (4) Form protoplasts by adding 4% (v/v) Rohament CL and 0.4% (v/v) Rohapect UF (AB Enzymes™, Darmstadt, Germany), stir manually with a serological pipette. (5) Transfer suspension to an Optimum Growth™ 5 L flask (preferably no more than 1.5 L in each 5 L flask, as higher volumes were found to result in suboptimal mixing of suspension during protoplastation) and incubate the suspension and cell wall degrading enzymes on an orbital shaker at 27° C. and 70-80 rpm for 2 hours. (6) During cell wall degradation, prepare a step-wise Percoll gradient in a 50 mL high-speed round bottom centrifuge tubes that comprises, from bottom to top, the following Percoll gradient layers: 70% (v/v, 5 mL), 50% (v/v, 7 mL), 40% (v/v, 7 mL), 30% (v/v, 3 mL), 15% (v/v, 3 mL) and 0% (3 mL). Composition of 0% and 70% Percoll gradient solutions are shown in Table 5 below; 15% to 50% gradient solutions are made by mixing the corresponding volumes of 0% and 70% gradient solutions. The gradient is started by adding 0% Percoll solution to the bottom of the tube and then, using a 25 mL serological pipette, adding to the bottom of the tube each successively heavier gradient layer solutions, directly underneath the next lightest layer. Depending on the packed cell volume (PCV 20-30%) of the main culture, 5-6 gradients are needed per liter of main culture.

TABLE 5

| | 0% Percoll Solution | 70% Percoll Solution |
|---|---|---|
| | (Evacuolation Buffer) | (Evacuolation Buffer) |
| Sorbitol | 0.7M | 0.7M |
| PIPES | 5 mM | 5 mM |
| MgCl2 | 20 mM | 20 mM |
| Water | To Final Volume | To 30% Final Volume |
| KOH | Adjust to pH 7.0 | 5 mM |
| | (5M KOH, about | (5M KOH added prior |
| | 2 mL/liter) | to Percoll) |
| Percoll | — | 70% v/v |

(7) Check the formation of protoplasts under the microscope (at least 100× magnification). At least 85% of the used cells should be present as single protoplasts with a spherical shape and a size of 50-80 μm. There will still be some cell aggregates present. (8) Pass protoplasts carefully through a 100 μm nylon mesh. (9) Harvest protoplasts by centrifugation (in 50 mL conical tubes) at room temperature and 120×g for 5 minutes (using slow deceleration). Protoplasts will pellet at the bottom of the 50 mL conical tubes. (10) Decant the major part of the supernatant consisting of the culture medium and the cell wall degrading enzymes. Remaining volume (protoplast pellet plus residual supernatant) should be around 5 mL. (11) Aspirate protoplast pellet and residual supernatant using a 25 mL serological pipette and transfer the suspension on the top of the Percoll gradient using a 25 mL serological pipette. (12) Low-density vacuoles are removed from protoplasts by centrifugation at 20° C., 10,000×g for 1 hour (using slow rotor acceleration and deceleration setting). Removed vacuoles float to the top of the Percoll gradient. Partial evacuolated protoplasts or damaged protoplasts concentrate at the 15/30% and 30/40% Percoll interface. Evacuolated protoplasts (miniprotoplasts) concentrate at the 40/50% and 50/70% Percoll interface. (13) Remove the upper layers (0-40% Percoll) using a vacuum aspiration system until reaching evacuolated protoplasts layer at the 40/50% and 50/70% Percoll interface. (14) Carefully pipette evacuolated protoplasts into a new 50 mL conical tube using a 10 mL serological pipette. (15) Add at least four volumes (based on the volume of evacuolated protoplasts) of 0.7 M mannitol (mannitol dissolved in water) wash buffer and mix by inverting the tube several times. Lower mannitol to miniprotoplasts ratios tend to result in suboptimal pelleting in step 17 below due to residual Percoll). (16) Pass miniprotoplasts through a 50 μm mesh. (17) Check miniprotoplasts under microscope (at least 100× magnification). Miniprotoplasts should have a spherical shape without vacuoles and a size of about 25-35 μm. (18) To remove mannitol, centrifuge at room temperature, 100×g for 5 minutes. Decant as much supernatant as possible. (19) Resuspend pellet in 1 volume of TR buffer in a 50 mL conical tube. TR buffer: 30 mM HEPES-KOH (pH 7.6), 40 mM potassium glutamate, 0.5 mM magnesium glutamate, 2 mM dithiothreitol (DTT), supplemented with one tablet per 50 mL of Complete™ EDTA-free Protease Inhibitor Mixture (Roche Diagnostics, Mannheim, Germany). (20) Transfer miniprotoplasts into a pre-chilled 15 mL Dounce tissue grinder and disrupt the miniprotoplasts by 30 strokes on ice. (21) After homogenization add another 2 volumes of TR buffer. (22) Centrifuge the homogenized sample at 4° C. and 500×g for 5 minutes to produce two phases: an upper liquid phase (=soybean lysate) and a lower solid phase containing the membranes and nuclei (if no visible pellet is apparent, add 1 mL additional TR buffer, stir with a clean tip smoothly in the upper phase and repeat centrifugation). (23) Transfer the upper phase to a new 50 mL conical tube and discard the pellet. (24) Add 1% (v/v) dimethyl sulfoxide (DMSO) and mix thoroughly by inverting and flipping the tube. (25) Prepare soybean cell-free lysate aliquots of 1 mL in 2 mL microcentrifuge tubes and freeze at −80° C.

Example 3: Protein Expression in Improved Soybean Cell-Free Lysate. Linearized plasmid template suitable for capped mRNA synthesis was generated by EcoRI digestion of pIVEX_GAAAGA_Omega_eYFP-His vector disclosed in U.S. Pat. No. 10,612,031. Linearized plasmid was purified using a PCR purification kit. Capped mRNA was prepared using HiScribe™ T7 High Yield RNA Synthesis Kit (New England Biolabs, Ipswich, MA) and m2^{7,3′-O}GP₃G (ARCA Cap Analog) from Jena Bioscience (Jena, Germany) using manufacturer recommended conditions and 1 μg of linearized plasmid DNA (Omega_Strep-eYFP) per 20 μL reaction. Synthesized mRNA was purified using DyeEx 2.0 Spin Kit from Qiagen (Germantown, MD USA) and the mRNA concentration was determined using a NanoDrop™ instrument from Thermo Fisher Scientific (Wilmington, DE USA).

Synthesis of eYFP protein was carried out using 5 μg of capped mRNA in the translation reaction components described in Tables 6, 7, and 8 below. Soybean cell-free lysates (“SBL”) prepared as described in Example 2 were used to set up in separate 50 μL reactions, incubated at 25° C. and 500 rpm for 18 h in Kuhner shaker. Protein yields were compared as between SBL taken from 40/50% gradient interface and the 50/70% gradient interface. The fluorescent signal from eYFP was quantified using a Synergy™ HT Multi-Mode Microplate Reader (Biotek™, Bad Friedrichshall, Germany) with 485/20 nm excitation and 528/20 nm emission filters. The quantity of eYFP was determined by generating a standard curve based on different concentrations of eYFP in SBL translation reactions without an mRNA template. The eYFP standard was produced using the tobacco BYL in vitro transcription-translation system, and purified via the Strep-tag by affinity chromatography using Strep-Tactin™ XT (IBA Lifesciences, Goettingen, Germany). The concentration of purified eYFP was determined using a colorimetric assay. Bradford (1976) Anal. Biochem. 72: 248-54.

TABLE 6

| Translation Reaction Components | Volume (μL) |
|---|---|
| H₂O | 4.5 |
| Translation Reaction Buffer (Table 7) | 6.2 |
| 50 mM Magnesium glutamate | 0.43 |
| 4000 mM Potassium glutamate | 0.33 |
| 10x Translation Mix (Table 8) | 5 |
| Creatine phosphokinase (10 mg/mL) | 1 |
| mRNA | 2.5 (5 μg) |
| SBL (W82) | 30 |
| Total | 50 |

TABLE 7

| Translation Reaction Buffer | Concentration/Amount | |
|---|---|---|
| HEPES-KOH, pH 7.6 | 30 | mM |
| Potassium glutamate | 60 | mM |
| Magnesium glutamate | 0.5 | mM |
| DTT | 2 | mM |

| | |
|---|---|
| Protease Inhibitor | 1 Tablet of complete, EDTA-free |
| | protease inhibitor per 50 ml |

TABLE 8

| 10x Translation Mix | Concentration | |
|---|---|---|
| ATP | 2.5 | mM |
| GTP | 0.33 | mM |
| Creatine-P | 380 | mM |
| Amino acids | 1.0 | mM |
| Spermine | 1.2 | mM |

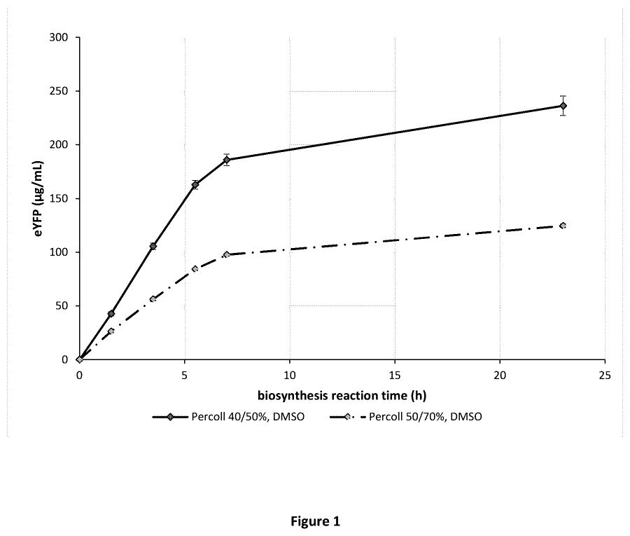

Protein synthesis results are shown in FIG. 1 (mean and standard deviation calculated based on three independent translation experiments). The results demonstrate that highest expression (˜230 mg/L) was obtained with SBL isolated from 40/50% gradient interface as compared to ˜125 mg/L from 50/70% gradient interface. Results in FIG. 1 also show that protein expression in soybean cell-free lysate increased linearly for nearly 6 hours.

Example 4: Flexibility of Soybean Cell-Free Lysate Expression System. Cell-free soybean lysate (SBL) was produced as described in Example 2 and was tested for its ability to express ten different target proteins shown in Table 9. Target proteins were fluorescently labeled during translation using FluoroTect™ system from Promega (Madison, WI USA). For each sample 20 μL of the reaction mix was loaded on a 4-12% (w/v) SDS-PAGE gradient gel, and protein products were visualized by fluorography. Distinct bands of varying strength migrated at the expected size for the ten target proteins as well as for control protein Strep-eYFP. Very strong bands were produced corresponding to Cry3A, Trap8Vip3A, Vip3A, and Cry6A proteins (as well as for control Strep-eYFP).

TABLE 9

| Protein | mW (kDa) | Lysine Residues | SDS-PAGE Band |
|---|---|---|---|
| 21333 | 130 | 30 | Yes |
| 22807 | 71 | 13 | Yes |
| AAD-12 | 32 | 6 | Yes |
| Cry1F | 130 | 34 | Yes |
| Cry2A | 71 | 8 | Weak |
| Cry3A | 73 | 33 | Strong |
| Trap8Vip3A | 95 | 69 | Strong |
| Vip3A | 88 | 63 | Strong |
| Cry6A | 54 | 37 | Strong |
| 17912 | 37 | 20 | Yes |
| Strep-eYFP | 29 | 21 | Strong |

Example 5: Soybean Cell-Free Lysate Expression System for Ranking Gene Variants. The cell-free soybean lysate (SBL) disclosed herein was used to assess expression levels of gene variants prior to stable plant transformation. Eleven constructs (Table 10) were created to test expression of four protein coding sequences with different GC content in the uncoupled transcription-translation system disclosed herein. Protein product was analyzed by SDS PAGE as in Example 4. A distinct band migrating at the expected size was observed for each target protein and their relative expression was ranked on a scale of 1-3 as shown in Table 10. Two isoforms of one gene (Cry1Ea) were expressed, a full-length (Fl) and a truncated (Tr) version.

TABLE 10

| | GC content | MW | Lysine | |
|---|---|---|---|---|
| Protein CDS | (% total) | (kDa) | Residues | Rank |
| TrCry1Ca_117306 | 37.5 | 70 | 6 | 2 |
| (Low GC) | | | | |
| TrCry1Ca_117333 | 43.6 | 70 | 6 | 1 |
| (High GC) | | | | |
| TrCry1Ca_117335 | 58.0 | 70 | 6 | 3 |
| (Very High GC) | | | | |
| Cry2Aa_117305 | 36.1 | 71 | 9 | 3 |
| (Low GC) | | | | |
| Cry2Aa_1173332 | 41.6 | 71 | 9 | 1 |
| (Medium GC) | | | | |
| Cry2Aa_117336 | 55.6 | 71 | 9 | 2 |
| (Very High GC) | | | | |
| Vip3Ab_117383 | 41.5 | 88 | 63 | 1 |
| (Medium GC) | | | | |
| Vip3Ab_117363 | 45.9 | 88 | 63 | 2 |
| (High GC) | | | | |
| TrCry1Ea_117376 | 43.1 | 68 | 6 | 1 |
| (Medium GC) | | | | |
| TrCry1Ea_117357 | 48.5 | 68 | 6 | 2 |
| (High GC) | | | | |
| FlCry1Ea_117357 | 48.8 | 130 | 37 | 3 |
| (High GC) | | | | |

Example 6: Preparation of Maize Protoplast Cell Lysate. Zea mays cv. Black Mexican Sweet (BMS) maize cell line that is not recalcitrant to protoplast formation was selected. BMS callus was used to establish a suspension culture in modified Murashige+Skoog/Gamborg B5 medium supplemented with coconut water (“BMS medium”, Table 11). Routine BMS cultures were cultivated by adding 1000 (v/v) PCV inoculant to 50 mL BMS medium, and growing for 7 days at 26° C. 140 rpm using a 16 h/8 h light/dark cycle in a 100 mL Erlenmeyer flask with aluminum foil cap. Larger routine culture volumes can be grown in 200 mL culture in 500 mL Erlenmeyer flask. Precultures were grown by inoculating 10% (v/v) PCV (from routine cultivation) into 400 mL of BMS medium and then incubating at 26° C., with shaking at 140 rpm, using a 16 hour/8 hour light/dark cycle for 7 days.

TABLE 11
BMS medium

| | | |
|---|---|---|
| Murashige + Skoog (MS) salts | 4.3 | g/L |
| Sucrose | 30 | g/L |
| Gamborg B5 vitamins | 0.112 | g/L |
| 2,4-D | 1 | mg/L |

| | |
|---|---|
| Coconut water | 5% (v/v) |

pH to 5.8 with 1M KOH/NaOH

To evaluate protoplast formation, main culture was grown in two 1 L Erlenmeyer flasks, each containing 400 mL BMS medium that was inoculated with preculture to produce 10% (v/v) PCV starting main culture. This main culture was incubated 26° C., with shaking at 140 rpm, using a 16 hour/8 hour light/dark cycle for 7 days to reach a final PCV of 20-25%0 (v/v). Cells were digested directly in the cultivation medium using 3.500 (v/v) Rohament® CL and 0.2% (v/v) Rohapect® UF (pectinases and arabanases). Osmolarity was adjusted by addition of 360 mM mannitol. Cells were almost completely converted to protoplasts, and little cell debris was observed.

For lysate production, BMS cell cultivation was scaled up by cultivating cultures as described above in a 1 L Erlenmeyer flasks to a PCV of 18% (v/v) and BMS cells were subjected to enzymatic treatment in cultivation medium as described for preculture.

Protoplasts from scale up preparation were layered onto a density gradient consisting of 0/15/30/40/50% (v/v) (each 7 mL) Percoll® media (GE Healthcare, Munich, Germany) in a 50 mL polypropylene tube (Greiner Bio-One™, Frickenhausen, Germany) and centrifuged at 6,800×g for 1 hour in a swinging-bucket rotor (JS-5.3, Beckmann-Coulter™, Krefeld Germany). Evacuolated protoplasts were recovered from between the 30/40% (v/v) Percoll layers and suspended in 3-3.5 volumes of TR buffer (30 mM HEPES-KOH (pH 7.4), 60 mM potassium glutamate, 0.5 mM magnesium glutamate, 2 mM DTT), supplemented with one tablet per 50 mL of Complete™ EDTA-free Protease Inhibitor Mixture (Roche Diagnostics, Mannheim, Germany). Protoplasts were then disrupted on ice using 15 strokes of a Dounce homogenizer (Braun, Melsungen, Germany), and the nuclei and non-disrupted cells were removed by centrifugation at 500×g for 10 minutes at 4° C. Cell lysate activity was assessed in an uncoupled transcription-translation reaction.

Example 7: Uncoupled Transcription Translation Reaction. Eleven expression constructs containing T7 promoter with different combinations of a 5′ untranslated region (5′ UTR) and 3′ untranslated region (3′ UTR) were prepared and tested for expression of yellow fluorescent protein (eYFP) from a streptavidin-eYFP fusion coding region (STREP-eYFP). The pF3A_Strep-eYFP vector is based on commercial pF3A vector (Promega™, Madison, WI USA) that uses Barley yellow dwarf virus (BYDV) UTRs. The other vectors use the pIVEX_GAAAGA_Omega_eYFP-His vector backbone disclosed in U.S. Pat. No. 10,612,031 in which all 5′ UTRs include the sequence GAAAGA upstream of initial GUA triplet. Vectors are schematically shown in Table 12.

TABLE 12

| | Pro- | | Coding | |
|---|---|---|---|---|
| Vector | moter | 5′ UTR | Region | 3′ UTR |
| pF3A_Strep- | T7 | BYDV | STREP- | BYDV |
| eYFP | | | eYFP | |
| Omega_Strep- | T7 | GAAAGA_ | STREP- | TMV |
| eYFP | | Omega | eYFP | |
| AtUbi10_Strep- | T7 | GAAAGA_ | STREP- | TMV |
| eYFP | | AtUbi10 | eYFP | |
| CHS_Strep- | T7 | GAAAGA_ | STREP- | TMV |
| eYFP | | CHS | eYFP | |
| 3xArc1_Strep- | T7 | GAAAGA_ | STREP- | TMV |
| eYFP | | 3xArc1 | eYFP | |
| PH_Strep- | T7 | GAAAGA_ | STREP- | TMV |
| eYFP | | PH | eYFP | |
| TEV_Strep- | T7 | GAAAGA_ | STREP- | TMV |
| eYFP | | TEV | eYFP | |
| Omega_oKSdi_ | T7 | GAAAGA_ | STREP- | TMV |
| Strep-eYFP | | Omega_oKSdi | eYFP | |
| BYDV_Strep- | T7 | GAAAGA_ | STREP- | BYDV |
| eYFP | | BYDV | eYFP | |
| MNeSV_Strep- | T7 | GAAAGA_ | STREP- | MNeSV |
| eYFP | | MNeSV | eYFP | |

Plasmid vectors were linearized by digestion with EcoRI endonuclease and then purified with PCR purification kit. Capped mRNA was prepared using HiScribe™ T7 High Yield RNA Synthesis Kit (New England Biolabs, Ipswich, MA) and m2^{7,3′-O}GP₃G (ARCA Cap Analog) from Jena Bioscience (Jena, Germany) using manufacturer recommended conditions and 1 μg of linearized plasmid DNA (Omega_Strep-eYFP) per 20 μL reaction. Synthesized mRNA was purified using DyeEx 2.0 Spin Kit from Qiagen (Germantown, MD USA) and the mRNA concentration was determined using a NanoDrop™ instrument from Thermo Fisher Scientific (Wilmington, DE USA).

Synthesis of eYFP protein was carried out in translation reactions using the BMS cell lysate and 5 μg of capped mRNA prepared as described above. Each translation reaction in a 50 μL total volume was incubated at 25° C. and 80% humidity with shaking at 500 rpm for 16 hours in Kuhner shaker (shaking diameter set to 12.5 mm). Components of translation reaction are described in Table 13. 10× Translation mix comprised ATP (2.5 mM), GTP (0.22 mM), creatine phosphate (380 mM), amino acids (1.0 mM), and spermine (1.2 mM). Translation (TR) buffer comprised HEPES-KOH, pH 7.6 (30 mM), Potassium glutamate (40 mM), Magnesium glutamate (0.5 mM), DTT (2 mM), and 1 tablet of complete, EDTA-free Protease Inhibitor per 50 mL.

TABLE 13

| Component | Working Conc. | | Final Conc. | | Volume or Amount | |
|---|---|---|---|---|---|---|
| H₂O | | | | | (as needed to 50 μL) | |
| TR buffer | | | | | 6.3 | μL |
| Mg Glutamate | 50 | mM | 0.4 | mM | 0.4 | μL |
| K Glutamate | 4000 | mM | 24 | mM | 0.3 | μL |
| 10x Translation mix | 10x | | 1x | | 5 | μL |
| Creatine kinase | 5 | mg/mL | 0.1 | mg/mL | 1 | μL |

| BMSL | 100% | 60% | | 30 | μL |
|---|---|---|---|---|---|
| mRNA, capped | | 0.1 | μg/μL | (5 | μg) |

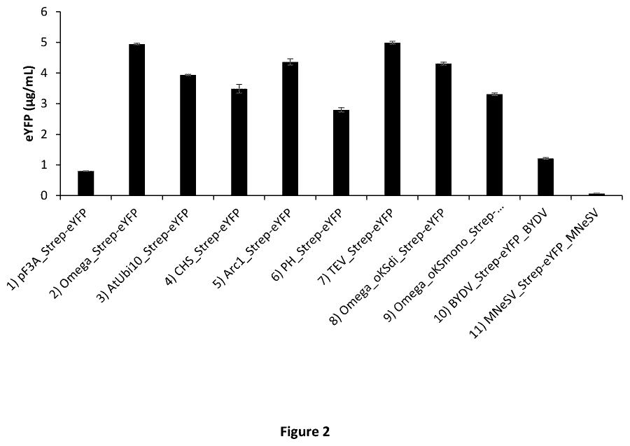

FIG. 2 shows the average and standard deviation of three independent translation experiments to measure the expression levels of eYFP from mRNA synthesized by each of the constructs in Table 10. The highest expressing construct (Omega_Strep-eYFP) produced an average expression level of about 5 μg per mL of lysate after 4.5 hours of expression. Longer incubation times did not lead to higher yields (data not shown).

Example 8: Use of Alternative Gradients Media for Protoplast Preparation. The Percoll gradient layer containing protoplasts used for translation in Example 6 was analyzed and found to include some non-evacuolated protoplasts and cell debris, in addition to protoplasts. Therefore, different density gradient media were tested.

The preparation of BMS protoplasts for lysate as described in Example 4 was modified as follows. After cell wall digestion, protoplasts were harvested by centrifugation in 50 mL conical tubes, at room temperature, at 140×g for 5 minutes (using slow deceleration). Protoplasts were then resuspended in 8-10 volumes of buffer containing 500 mM sucrose, 1 mM CaCl₂), and 5 mM MES-KOH pH 6.0. Resuspended protoplasts were “floated” by centrifugation at 140×g for 5 minutes. The resulting upper layer containing protoplasts was collected and protoplasts were washed once with 500 mM sorbitol, 1 mM CaCl₂), and 5 mM MES pH 6.0. Then the protoplasts were underlayed with the sucrose density gradient layers (from top to bottom): 0/250/500/800/1000 mM (5/5/5/7/5 mL) shown in Table 14. The gradient was centrifuged at 6,800×g for 1 hour and evacuolated protoplasts were mostly found between the 800/1000 mM sucrose layers.

TABLE 14

| | | | | | | | | | | |
|---|---|---|---|---|---|---|---|---|---|---|
| Sucrose | 1000 | mM | 800 | mM | 500 | mM | 250 | mM | 0 | mM |
| Sorbitol | 1000 | mM | 1000 | mM | 1000 | mM | 1000 | mM | 1000 | mM |
| CaCl₂ | 1 | mM | 1 | mM | 1 | mM | 1 | mM | 1 | mM |
| MES-KOH | 5 | mM | 5 | mM | 5 | mM | 5 | mM | 5 | mM |

| | | | | | |
|---|---|---|---|---|---|
| pH 6.0 | | | | | |
| Water | to 5 mL | to 7 mL | to 5 mL | to 5 mL | to 5 mL |

Lysate was prepared from BMS evacuolated protoplasts as described above and were evaluated for eYFP expression using the 11 expression constructs shown in Table 12. The AtUbi10_Strep-eYFP and 3xArc1_Strep-eYFP vectors produced the highest eYFP production: about 12 μg eYFP per mL lysate after 4 hours (data not shown). Longer incubation times did not lead to significantly increased eYFP yields.

Because sucrose gradient did not improve protein expression as much as desired, a density gradient based on iohexol (trade name Nycodenz®) was tested. Iohexol is a non-ionic tri-iodinated derivative of benzoic acid with three aliphatic hydrophilic side chains; its systematic name is Nico5-(N-2,3-dihydroxypropylacetamido)-2,4,6-tri-iodo-N—N′-bis(2,3-dihydroxypropyl) isophthalamide and molecular weight is 821.

In Nycodenz® test, a BMS 400 mL culture volume with a PCV of 20% (w/v) was digested directly in cultivation medium with a combination of 4% (v/v) Rohament® CL, 0.4% (v/v) Rohapect© UF, 400 mM sorbitol, 1 mM CaCl₂) directly in the fermentation medium. Protoplasts were harvested by centrifugation at 140×g for 5 minutes and underlayed with density gradient (from top to bottom) 0/5/10/15/20% (w/v) (3/5/5/5/5 mL) Nycodenz® solutions shown in Table 15. Density gradient was centrifuged at 10,000×g for 1 hour, and evacuolated protoplasts were mainly found between the 10/15% (w/v) Nycodenz® layers. Evacuolated protoplasts were washed in 0.7 M mannitol buffer with 1 mM CaCl₂) to remove Nycodenz®. Cell debris was removed by passing the evacuolated protoplasts through a 30 μm nylon mesh. Evacuolated protoplasts were resuspended in 1 volume of TR buffer (see Table 16) and disrupted using a Dounce tissue grinder. After Dounce homogenization, another 2 volumes of TR buffer were added. The final lysate was frozen with 1% (v/v) DMSO as cryoprotectant. Approximately 2.3 mL of maize lysate was obtained from the starting 400 mL cell culture.

TABLE 15

| Nycodenz ® | 20% | (w/v) | 15% | (w/v) | 10% | (w/v) | 5% | (w/v) | 0% | |
|---|---|---|---|---|---|---|---|---|---|---|
| Sorbitol | 1000 | mM | 1000 | mM | 1000 | mM | 1000 | mM | 1000 | mM |
| CaCl₂ | 1 | mM | 1 | mM | 1 | mM | 1 | mM | 1 | mM |
| MES-KOH | 5 | mM | 5 | mM | 5 | mM | 5 | mM | 5 | mM |

| | | | | | |
|---|---|---|---|---|---|
| pH 6.0 | | | | | |
| Water | to 5 mL | to 5 mL | to 5 mL | to 5 mL | to 3 mL |

TABLE 16

| | Stock | Final | |
|---|---|---|---|
| HEPES-KOH pH 7.6 | 1000 mM | 30 | mM |
| Potassium glutamate | 4000 mM | 40 | mM |
| Magnesium glutamate | 1000 mM | 0.5 | mM |
| DTT | 1000 mM | 2 | mM |

| | |
|---|---|
| Complete ™ Protease | 1 tablet per |
| inhibitor | 50 mL |

Nycodenz® gradient cell lysate was tested using the same uncoupled transcription-translation reaction described above using capped mRNA from the Omega_Strep-eYFP vector as template. The translation reactions produced about 60 μg protein per mL translation reaction. Thus maize cell lysate prepared using Nycodenz® gradients for evacuolation achieved the highest expression levels among all of the methods tested and was used in the following optimized procedure:

(1) Harvest cell suspension into a 1 L beaker and determine the volume of the cell suspension. (2) Weigh sorbitol to final concentration of 0.4 M of the cell suspension and then dissolve sorbitol completely in the cell suspension by manual stirring (e.g. with a serological pipette). (3) Add 1 mM CaCl₂) (stabilizes membranes) using a 1 M CaCl₂) stock solution in distilled water and stir manually with a serological pipette until an even distribution is obtained. (4) Form protoplasts by adding 4% (v/v) Rohament CL and 0.4% (v/v) Rohapect UF, stir manually with a serological pipette until an even distribution is obtained. (5) Transfer suspension to Optimum Growth™ 5 L flask (preferably no more than 1.5 L in each flask, as higher volumes can lead to suboptimal mixing of the suspension during protoplastation) and incubate the Optimum Growth™ 5 L flask containing the suspension and cell wall degrading enzymes on an orbital shaker at 27° C. and 70-80 rpm for 2 hours. (6) Check the formation of protoplasts under the microscope (at least 100× magnification). At least 85% of the used cells should be present as single protoplasts with a spherical shape and a size of 40-80 μm. There will still be some cell aggregates present. (7) Pass protoplasts carefully through a 100 μm nylon mesh. If the filter becomes clogged, clean the filter with distilled water. Remove residual water by tapping the filter on the bench. (8) Harvest protoplasts by centrifugation (in 50 mL conical tubes) at room temperature and 140×g for 5 minutes (using slow deceleration). Protoplasts will pellet at the bottom of the 50 mL conical tubes. (9) Decant major part of supernatant consisting of culture medium and cell wall degrading enzymes. Remaining volume (protoplast pellet plus residual supernatant) should be about 5 mL. (10) Aspirate protoplast pellet and residual supernatant using a 25 mL serological pipette and transfer the suspension to a 50 mL high-speed round bottom centrifuge tube (each 15 mL per tube). (11) Prepare gradient using a multichannel peristaltic pump (flow rate 10 mL/min) or 25 mL serological pipette. Start with the 0% Nycodenz® solution (w/v, 3 mL), then proceed to successively pump the other solutions carefully below the previous one on the bottom of the 50 mL high-speed centrifuge tubes in the following amounts: 5% (w/v) 5 mL, 10% (w/v) 7 mL, 15% (w/v), 7 mL, 20% (w/v), 7 mL as shown in Table 17. Depending on the packed cell volume (PCV 20-25%) of the main culture 5-6 gradients should be used per liter of main culture.

TABLE 17

| Nycodenz ® | 20% | (w/v) | 15% | (w/v) | 10% | (w/v) | 5% | (w/v) | 0% | |
|---|---|---|---|---|---|---|---|---|---|---|
| Sorbitol | 1000 | mM | 1000 | mM | 1000 | mM | 1000 | mM | 1000 | mM |
| CaCl₂ | 1 | mM | 1 | mM | 1 | mM | 1 | mM | 1 | mM |
| MES-KOH | 5 | mM | 5 | mM | 5 | mM | 5 | mM | 5 | mM |
| pH 6.0 | | | | | | | | | | |
| Layer vol. | 7 | mL | 7 | mL | 7 | mL | 5 | mL | 3 | mL |

The 0% Nycodenz® evacuolation buffer was sterilized by filtration using a 0.2 μm bottle top filter, and can be stored at 4° C. The Nycodenz®-containing gradient solutions were prepared by weighing appropriate Nycodenz® amount and adding distilled water to 90% of final volume (e.g., to 450 mL to prepare 0.5 L of 50% (w/v) Nycodenz® buffer). Nycodenz® was dissolved by heating to 50° C. After cooling to room temperature, 5 mM MES-KOH pH 6.0, 1 mM CaCl₂) and distilled water to 100% of the final volume were added. Solutions were sterilized by filtration using a 0.2 μm bottle top filter. Individual gradient layers were prepared by mixing corresponding volumes of 0% and 50% Nycodenz® buffers. The 50% (w/v) Nycodenz® buffer and individual Nycodenz® gradient layer can be stored at 4° C. The gradient, however, was prepared fresh, immediately before use.

(12) Low-density vacuoles are separated from protoplasts by centrifugation at 20° C. and 10,000×g for 1 hour using a swinging bucket rotor (select slow acceleration and deceleration of rotor in the centrifuge adjustments). Vacuoles float to the top of the Nycodenz® gradient. Partially evacuolated protoplasts or damaged protoplasts concentrate at the 0/5% (w/v) Nycodenz interface. Evacuolated protoplasts (i.e., miniprotoplasts) concentrate at the 5/10% (w/v) (or 10/15% (w/v)) Nycodenz® interface. (13) Remove the upper layers (0-5% (w/v) Nycodenz) using a vacuum aspiration system until reaching evacuolated protoplasts layer at the 5/10% (w/v) (or 10/15% (w/v)) Nycodenz interface. (14) Carefully pipette evacuolated protoplasts into a new 50 mL conical tube using a 10 mL serological pipette. (15) Add at least three volumes (based on the volume of evacuolated protoplasts) of 0.7 M mannitol wash buffer (containing 5 mM MES-KOH pH 6.0 and 1 mM CaCl₂)) (wash buffer was filter sterilized using 0.2 μm bottle top filter and can be stored at 4° C.). Miniprotoplasts and wash buffer are mixed by inverting the tube several times. (16) Pass miniprotoplasts carefully through a 50 μm nylon mesh. If filter becomes clogged, rinse the filter with distilled water. Remove residual water by tapping the filter on the bench. (17) Check miniprotoplasts under the microscope (at least 100× magnification). They should have a spherical shape without vacuoles and a size of ˜25-35 μm. (18) Remove mannitol by centrifuge at room temperature and 100×g for 5 minutes using a swinging bucket rotor, decant as much supernatant as possible. (19) All further steps are done on ice. (20) Resuspend pellet in 1 volume of TR buffer (Table 16) in 50 mL conical tube. Prepared TR buffer can be stored at −20° C.

(20) Transfer miniprotoplasts to a pre-chilled 15 mL Dounce tissue grinder and disrupt the miniprotoplasts by 30 strokes on ice. (21) After homogenization, add another 1.5 volumes of TR buffer and transfer disrupted miniprotoplasts to 50 mL conical tubes. (22) Membranes and nuclei are separated by centrifugation at 4° C. and 500×g for 5 minutes using a swinging bucket rotor. Two phases should be formed. An upper liquid phase (maize lysate) and a lower solid phase containing the membranes and nuclei. If there is no visible pellet, stir the upper phase smoothly, with a clean tip, and repeat centrifugation). (23) Transfer the upper phase to a new 15 mL conical tube and discard the pellet. (24) Add 1% (v/v) DMSO and mix thoroughly by inverting and flipping the tube. (25) Prepare aliquots of 0.5 mL in 2 mL microcentrifuge tubes and freeze at −80° C.

Example 9: Flexibility of Maize Cell-Free Lysate Expression System. Optimized Nycodenz® maize cell-free lysate system and protocol described in the foregoing example was tested for its ability to express ten different target proteins (Table 18). Reactions were carried out at 25° C. and 500 rpm for 4 hours. Target proteins were fluorescently labeled during translation using FluoroTect™ system from Promega (Madison, WI USA). For each sample 20 μL of the reaction mix was loaded on a 4-12% (w/v) SDS-PAGE gradient gel, and protein products were visualized by fluorography. A distinct band migrating at the expected size was observed for five out of ten target proteins as well as for control protein Strep-eYFP. Particularly strong bands were produced corresponding to AAD-12 and 17912 proteins.

TABLE 18

| Protein | mW (kDa) | Lysine Residues | SDS-PAGE Band |
|---|---|---|---|
| 21333 | 130 | 30 | — |
| 22807 | 71 | 13 | Yes |
| AAD-12 | 32 | 6 | Strong |
| Cry1F | 130 | 34 | — |
| Cry2A | 71 | 8 | — |
| Cry3A | 73 | 33 | Yes |
| Trap8Vip3A | 95 | 69 | Yes |
| Vip3 A | 88 | 63 | — |
| Cry6A | 54 | 37 | — |
| 17912 | 37 | 20 | Strong |
| Strep-eYFP | 29 | 21 | Strong |

Example 10: Maize Cell-Free Lysate Expression System for Ranking Gene Variants. The maize cell-free lysate system disclosed herein was used to assess expression levels of gene variants prior to stable plant transformation. Eleven constructs (Table 19) were created to express four gene coding sequences with different GC content in the uncoupled transcription-translation system disclosed herein. Protein product was analyzed by SDS PAGE as in Example 9. A distinct band migrating at the expected size was observed for each of ten of eleven target proteins and their relative expression was ranked on a scale of 1-3 as shown in Table 19. Two isoforms of one gene (Cry1Ea) were expressed, a full-length (Fl) and a truncated (Tr) version.

TABLE 19

| | GC content | MW | Lysine | |
|---|---|---|---|---|
| Protein CDS | (% total) | (kDa) | Residues | Rank |
| TrCry1Ca_117306 | 37.5 | 70 | 6 | 2 |
| (Low GC) | | | | |
| TrCry1Ca_117333 | 43.6 | 70 | 6 | 2 |
| (High GC) | | | | |
| TrCry1Ca_117335 | 58.0 | 70 | 6 | 3 |
| (Very High GC) | | | | |
| Cry2Aa_117305 | 36.1 | 71 | 9 | 3 |
| (Low GC) | | | | |
| Cry2Aa_1173332 | 41.6 | 71 | 9 | 1 |
| (Medium GC) | | | | |
| Cry2Aa_117336 | 55.6 | 71 | 9 | 2 |
| (Very High GC) | | | | |
| Vip3Ab_117383 | 41.5 | 88 | 63 | 1 |
| (Medium GC) | | | | |
| Vip3Ab_117363 | 45.9 | 88 | 63 | 2 |
| (High GC) | | | | |
| TrCry1Ea_117376 | 43.1 | 68 | 6 | 1 |
| (Medium GC) | | | | |
| TrCry1Ea_117357 | 48.5 | 68 | 6 | 2 |
| (High GC) | | | | |
| FlCry1Ea_117357 | 48.8 | 130 | 37 | 3 |
| (High GC) | | | | |

Strongest bands were observed for target genes with medium GC content. Indicating BMSL is suitable for screening sequence variants. Similarly, truncated version of Cry1Ea expressed much better in the BMSL system compared to the full-length version. These results show that the cell-free maize lysate disclosed herein can be used to express a variety of insect resistance protein coding sequences to assess the impact of coding sequence GC content and encoded protein length on expression level. The results also support the use of the cell-free maize lysate disclosed herein to predict the impact of different coding sequence isoforms on transgenic expression levels in planta.

1. A method for synthesizing at least one polypeptide, the method comprising providing a soybean or maize cellular lysate;
combining the lysate with exogenous nucleic acid template and exogenous amino acids to form a reaction volume; and
synthesizing a polypeptide encoded by the exogenous nucleic acid template in the reaction volume.

2. The method according to claim 1, further comprising:
preparing protoplasts from soybean or maize cells;
evacuolating the protoplasts to form miniprotoplasts;
disrupting the membranes of miniprotoplasts to form a cellular lysate; and
removing nuclei from the cellular lysate prior to combining the cellular lysate with the exogenous nucleic acid.

3. The method of claim 1, wherein the cellular lysate is prepared from homogenized soybean miniprotoplasts.

4. The method of claim 3, wherein the method comprises synthesizing about 100 μg/mL or more of the encoded polypeptide, wherein mL refers to the reaction volume.

5. The method of claim 3, wherein the method comprises synthesizing about 200 μg/mL or more of the encoded polypeptide, wherein mL refers to the reaction volume.

6. The method of claim 1, wherein the cellular lysate is prepared from homogenized maize miniprotoplasts.

7. The method of claim 6, wherein the method comprises synthesizing about 1 μg/mL or more of the encoded polypeptide, wherein mL refers to the reaction volume.

8. The method of claim 6, wherein the method comprises synthesizing about 3 μg/mL or more of the encoded polypeptide, wherein mL refers to the reaction volume.

9. The method of claim 6, wherein the method comprises synthesizing about 5 μg/mL or more of the encoded polypeptide, wherein mL refers to the reaction volume.

10. A method of screening nucleic acid coding sequences for transgenic expression in maize or soybean, the method comprising:
providing a plurality of nucleic acids, each comprising a polypeptide coding sequence;
combining each nucleic acid of the plurality with a cellular lysate and synthesizing its encoded polypeptide in accordance with the method of claim 1;
evaluating each of the synthesized polypeptides to determine the expression profile of the nucleic acid sequence encoding the polypeptide; and
selecting one or more of the nucleic acid coding sequences for transgenic expression in maize or soybean based on the expression profile of the polypeptide encoded by selected one or more nucleic acid sequences.

11. The method of claim 11, further comprising transforming maize or soybean plant tissue with one or more of the selected nucleic acid coding sequence for transgenic expression and thereby generate transgenic maize or soybean plant tissue, wherein the cellular lysate and transgenic tissue are of the same plant species.

12. The method of claim 12, further comprising regenerating a transgenic plant from the transgenic maize or soybean plant tissue.

13. A method of preparing cellular lysate for synthesis of a biopolymer, the method comprising:
preparing protoplasts from soybean or maize cells;
evacuolating the protoplasts to form miniprotoplasts;
disrupting the membranes of miniprotoplasts to form a lysate; and
removing nuclei from the homogenized lysate to form a cellular lysate for synthesis of a biopolymer.

14. The method of claim 13, wherein the protoplasts are prepared from soybean cells and the soybean protoplasts are evacuolated and miniprotoplasts are isolated by Percoll gradient separation.

15. The method of claim 14, wherein the protoplasts are prepared from maize cells and the y maize protoplasts are evacuolated and miniprotoplasts are isolated by iohexol gradient separation.

16. The method of claim 13, wherein the miniproplasts membranes are disrupted and homogenized in a buffer comprising HEPES, potassium glutamate, magnesium glutamate, redox reagent and protease inhibitor.

17. The method of claim 13, wherein the method further includes adding polymer template and monomeric units of the polymer.

18. A kit or system for synthesis of a biopolymer, the system comprising a reaction volume comprising:
cellular lysate prepared from maize or soybean miniprotoplasts; and
buffer for in vitro transcription and/or in vitro translation.

19. A kit or system for synthesis of a biopolymer, the system comprising a reaction volume comprising:
a cellular lysate from maize or soybean prepared in accordance with claim 13; and
buffer for in vitro transcription and/or in vitro translation.

20. A kit according to claim 18, wherein the maize or soybean cellular lysate and the buffer are each provided in a separate volume container.
